(19) United States
(12) Patent Application Publication
(10) Pub. No.: US 2026/0216741 A1
(43) Pub. Date: Jul. 30, 2026

(54) COMMUNICATION ENABLED DISPOSABLE CARTRIDGE FOR ELECTROSTATIC SPRAYING

(71) Applicant: Octet Medical, Inc., San Diego, CA (US)

(72) Inventors: Clifford A. Wright, San Diego, CA (US); Benjamin Kinney, San Diego, CA (US); Kyle Elsabee, San Diego, CA (US)

(73) Assignee: Octet Medical, Inc., San Diego, CA (US)

(21) Appl. No.: 19/572,768

(22) Filed: Mar. 19, 2026

Related U.S. Application Data
(63) Continuation-in-part of application No. 19/407,735, filed on Dec. 3, 2025.
Application No. 19/407,735 is a continuation of application No. 18/374,647, filed on Sep. 28, 2023, now Pat. No. 12,521,737.
Application No. 18/374,647 is a continuation of application No. 18/110,854, filed on Feb. 16, 2023, now Pat. No. 11,806,740.
(60) Provisional application No. 63/411,334, filed on Sep. 29, 2022.

(51) Int. Cl. B05B 5/16; A61M 35/00

(52) U.S. Cl. CPC B05B 5/1691; A61M 35/003; B05B 5/1675

(57) ABSTRACT

Systems and methods are disclosed for operating disposable fluid delivery system for an electrostatic applicator, which can include establishing communication between a communication interface system of a disposable cartridge and memory of the electrostatic applicator; verifying authenticity of an encrypted data set stored on the communication interface system; and configuring operating parameters that comprise at least one of at least one spray-generation parameter and/or at least one electrostatic-charging parameter based at least on the encrypted data set.

### CROSS-REFERENCE TO RELATED APPLICATION

This application claims priority to and the benefit of U.S. Provisional Patent Application Ser. No. 63/411,334, filed Sep. 29, 2022, and to U.S. Non-Provisional patent application Ser. No. 18/110,854, filed Feb. 16, 2023, now U.S. Pat. No. 11,806,740, issued Nov. 7, 2023, and to of U.S. Continuation Non-Provisional patent application Ser. No. 18/374,647, filed Sep. 28, 2023, now U.S. Pat. No. 12,521,737 issued Jan. 23, 2026 and is a Continuation-in-Part of U.S. Non-Provisional application Ser. No. 19/407,735, filed Dec. 3, 2025. The contents of each of the foregoing applications are hereby incorporated by reference in their entirety as if fully set forth herein.

### FIELD

The disclosure relates to devices, systems, and methods for electrostatic application of treatment solutions to a target site. More specifically, the devices, systems, and methods are directed towards communication-enabled disposable cartridges and/or applicators that store encrypted operating parameters, authenticate cartridge identity, and configure spray-generation and electrostatic-charging settings.

### BACKGROUND

Healthcare delivery often requires precise, localized application of treatment solutions, including antiseptics, anesthetics, analgesics, biologics, and other therapeutics. Conventional modalities such as oral or systemic administration can be inefficient for site-specific therapy and may expose patients to unnecessary systemic burden. Alternative vehicles (e.g., gels, films, or dressings) can improve localization but may introduce manufacturing complexity, sterilization challenges, and variability in dosing uniformity and adherence to irregular anatomical surfaces.

Electrostatic spraying has emerged as a technique for creating fine, charged droplets that can deposit uniformly on complex geometries while reducing run-off and improving coverage efficiency. However, electrostatic performance is highly sensitive to formulation-specific factors such as viscosity, conductivity, surface tension, and dielectric properties. A single, static sprayer configuration rarely optimizes plume shape, droplet-size distribution, or charge-to-mass ratio across diverse treatment solutions. As a result, users are forced to manually tune operating parameters such as flow rate, pulse timing, air intake, and applied voltage, which can be time-consuming, inconsistent, and error-prone in clinical settings.

Moreover, conventional electrostatic sprayers often lack secure, interchangeable cartridge systems that can authenticate consumables, prevent counterfeit or incompatible use, and enforce single-use or limited-use protocols to support safety and regulatory compliance. Absent secure identification and authentication, devices cannot reliably retrieve formulation-specific operating parameters, leaving performance dependent on manual input. Likewise, many systems lack closed-loop sensing and data logging to adjust for environmental variation (e.g., ambient humidity, temperature) and device state (e.g., motor speed, airflow, applied voltage), or to provide post-procedural traceability.

This disclosure resolves these and other issues of the art.

### SUMMARY

The subject of this disclosure is an electrostatic applicator that utilizes a disposable cartridge to dispense a treatment solution, with a communication interface system (e.g., a near-field communication (NFC) integrated circuit) to authenticate the cartridge and automatically configure spray-generation and electrostatic-charging parameters based on the specific properties of the treatment solution.

In some examples, a disposable fluid delivery system for an electrostatic applicator is disclosed. The system can include a disposable cartridge containing and/or configured to receive a treatment solution and a nozzle assembly configured to dispense the treatment solution. A communication interface system, such as an NFC integrated circuit, can be positioned with the cartridge. The communication interface system can be an integrated circuit including memory that stores an encrypted data set including operating parameters that include at least one of at least one spray-generation parameter, at least one electrostatic-charging parameter, and an authenticity credential. The operating parameters can be programmed and/or tuned based at least in part on properties of the treatment solution of the cartridge. The memory can include instructions that, when executed by one or more processors, cause the communication interface system to exchange data with an electrostatic spray-generation subsystem, retrieve and decrypt the encrypted data set, authenticate the disposable cartridge based on its authenticity credential, and configure the electrostatic spray-generation subsystem in accordance with detected operating parameters of the encrypted data.

In some examples, the disposable cartridge is configured to receive a syringe containing the treatment solution.

In some examples, the disposable cartridge is removably attachable to a housing of the electrostatic applicator.

In some examples, the disposable cartridge includes a fluid reservoir containing a treatment solution.

In some examples, the disposable cartridge includes memory including information associated with operational parameters of the treatment solution.

In some examples, the cartridge or a syringe assembled with the cartridge is configured to be filled with the treatment solution.

In some examples, the instructions cause the communication interface system (e.g., the NFC integrated circuit in some aspects) to write, after a predetermined eject-mass or elapsed-time threshold is reached, a non-reversible usage flag to disable subsequent reuse of the cartridge.

In some examples, the NFC integrated circuit can be a one-time-programmable or write-once, read-many device that prevents alteration of the encrypted data set.

In some examples, the spray-generation parameter includes at least one of flow rate, pulse width, pulse frequency, and a fluidic pressure set-point.

In some examples, the electrostatic-charging parameter includes at least one of applied voltage magnitude, current limit, discharge waveform, and polarity.

In some examples, the NFC integrated circuit can be part of an NFC tag that includes an antenna configured to wirelessly communicate information.

In some examples, the instructions cause the NFC integrated circuit to select from a library of nozzle geometries optimized for different rheological profiles of one or more treatment solutions.

In some examples, the instructions cause the NFC integrated circuit to store nozzle-specific and material-specific parameters so that spray performance is tuned for a dispensing combination of nozzle and formulation parameters.

In some examples, the system further includes a plurality of sensors configured to measure operating parameters of the cartridge including at least one of ambient humidity, motor speed, applied voltage, intake of air, ambient temperature, and air-flow velocity.

In some examples, the instructions cause the NFC integrated circuit to dynamically adjust at least one of the spray-generation and electrostatic-charging parameters based on sensor feedback.

In some examples, the instructions cause the NFC integrated circuit to dynamically adjust at least one of motor speed, intake of air, and applied voltage based on retrieved information.

In some examples, the instructions cause the NFC integrated circuit to log cartridge identification, usage metrics, and environmental data to a tamper-resistant memory for post-procedural traceability.

In some examples, the system includes a portable, reusable electrostatic applicator having a handheld device housing with a motor configured to drive a piston, a voltage source, a high voltage module electrically connected to the voltage source, and a cartridge chamber. A disposable cartridge can be removably insertable into the cartridge chamber. The cartridge can include a nozzle assembly with a nozzle housing having an air supply port, a voltage port, and a delivery outlet, a cartridge housing at least partially enclosing the nozzle housing, a voltage wire, and a syringe, and a communication interface system, such as an NFC integrated circuit, with memory storing an encrypted data set of operating parameters that include at least one of a spray-generation parameter, an electrostatic-charging parameter, and an authenticity credential. The operating parameters can be programmed and/or tuned based at least in part on properties of the treatment solution. The instructions in the memory cause the communication interface system to exchange data with an electrostatic spray-generation subsystem, retrieve and decrypt the encrypted data set, authenticate the disposable cartridge based on its authenticity credential, and configure the electrostatic spray-generation subsystem in accordance with detected operating parameters of the encrypted data.

In some examples, the system further includes a graphical user interface that visually indicates at least one of cartridge authentication, status of parameter execution, and user intervention status.

In some examples, the system further includes a graphical user interface that visually displays a warning message.

In some examples, the system further includes a graphical user interface that visually presents information read from the communication interface system.

In some examples, the disposable cartridge is configured to receive a syringe containing the treatment solution.

In some examples, the disposable cartridge includes a fluid reservoir containing a treatment solution.

In some examples, the disposable cartridge includes memory including information associated with operational parameters of the treatment solution.

In some examples, the cartridge or a syringe assembled with the cartridge is configured to be filled with the treatment solution.

In some examples, the instructions cause the communication interface system to write, after a predetermined eject-mass or elapsed-time threshold is reached, a non-reversible usage flag to disable subsequent reuse of the cartridge.

In some examples, the communication interface system is a one-time-programmable or write-once, read-many device that prevents alteration of the encrypted data set.

In some examples, the spray-generation parameter includes at least one of flow rate, pulse width, pulse frequency, and a fluidic pressure set-point.

In some examples, the electrostatic-charging parameter includes at least one of applied voltage magnitude, current limit, discharge waveform, and polarity.

In some examples, the communication interface system can be an NFC integrated circuit that is part of an NFC tag that includes an antenna configured to wirelessly communicate information.

In some examples, the instructions cause the NFC integrated circuit to select from a library of nozzle geometries optimized for different rheological profiles of one or more treatment solutions.

In some examples, the instructions cause the NFC integrated circuit to store nozzle-specific and material-specific parameters so that spray performance is tuned for a dispensing combination of nozzle and formulation parameters.

In some examples, the system further includes a plurality of sensors configured to measure operating parameters of the cartridge including at least one of ambient humidity, motor speed, applied voltage, intake of air, ambient temperature, and air-flow velocity.

In some examples, the instructions cause the communication interface system to log cartridge identification, usage metrics, and environmental data to a tamper-resistant memory for post-procedural traceability.

In some examples, a method includes inserting a disposable cartridge into the electrostatic applicator, establishing communication between a communication interface system of the disposable cartridge and memory of the electrostatic applicator, verifying authenticity of an encrypted data set stored on the communication interface system, and configuring operating parameters that include at least one of at least one spray-generation parameter and/or at least one electrostatic-charging parameter based at least on the encrypted data set.

In some examples, the method further includes dispensing the treatment solution through a nozzle assembly of the disposable cartridge while applying electrostatic charge according to the configured operating parameters.

In some examples, the method includes monitoring, using one or more sensors of the disposable cartridge and/or the electrostatic applicator together with the communication interface system, cumulative dispensed mass or time of operation of treatment solution dispensed through the cartridge.

In some examples, the method includes measuring, using one or more sensors, at least one of ambient humidity, motor speed, applied voltage, intake air flow, ambient temperature, or air-flow velocity during dispensing through the disposable cartridge.

In some examples, the method includes, upon reaching a predefined end-of-life criterion, writing, using at least the communication interface system, an irreversible usage flag to inhibit further dispensing by the disposable cartridge.

In some examples, the method includes disabling operation or activation of the electrostatic applicator upon detection of the irreversible usage flag.

In some examples, the method includes adjusting the configured operating parameters during dispensing in response to environmental measurements of one or more sensors of the disposable cartridge and/or the electrostatic applicator.

In some examples, the method includes recording an event log that includes cartridge identifier, environmental conditions, and parameter adjustments, the event log being cryptographically signed and stored in secure memory.

In some examples, the method includes wirelessly communicating, using at least an NFC integrated circuit of an NFC tag of the communication interface system, cartridge operational information to a graphical user interface.

In some examples, the NFC integrated circuit is a one-time-programmable or write-once, read-many device that inhibits alteration of the encrypted data set after initial programming.

In some examples, the at least one spray-generation parameter includes at least one of flow rate, pulse width, pulse frequency, and fluidic pressure set-point.

In some examples, the method includes selecting, in response to data received from the NFC integrated circuit, a nozzle geometry from a library of nozzle geometries for differing profiles of treatment solutions.

In some examples, the method includes producing, using at least a nozzle assembly of the disposable cartridge, an electrostatically charged spray plume having a predetermined average droplet diameter and/or charge-to-mass ratio established by the configured spray-generation and electrostatic-charging parameters.

In some examples, configuring the operating parameters includes setting a voltage level that is modulated according to a flow rate commanded by the at least one spray-generation parameter.

In some examples, a non-transitory computer-readable medium stores instructions that, when executed by a processor, cause the processor to establish communication between a communication interface system of a disposable cartridge and memory of the electrostatic applicator, verify authenticity of an encrypted data set stored on the communication interface system, and configure operating parameters that include at least one of a spray-generation parameter and an electrostatic-charging parameter based at least in part on the encrypted data set.

In some examples, the communication between an NFC integrated circuit of the communication interface system and the memory is established by inserting the disposable cartridge into a chamber of the applicator.

In some examples, the encrypted data are derived from statistical optimization modeling based at least in part on droplet size distribution, plume shape, and/or mass-to-target metrics for a specific treatment solution.

In some examples, a treatment solution of the disposable cartridge includes a biologic suspension including at least one of monoclonal antibodies, polyclonal antibodies, peptides, and combinations thereof.

In some examples, a nozzle assembly orientation of the disposable cartridge is selected to accommodate a viscosity range between 1 cP and 10,000 cP.

In some examples, the method performed includes dispensing a treatment solution through a nozzle assembly of the disposable cartridge while applying electrostatic charge according to the configured operating parameters.

In some examples, the method performed includes monitoring, using one or more sensors of the disposable cartridge and/or the electrostatic applicator together with the NFC integrated circuit, cumulative dispensed mass or time of operation of treatment solution dispensed through the cartridge.

In some examples, the method performed includes measuring, using one or more sensors, at least one of ambient humidity, motor speed, applied voltage, intake air flow, ambient temperature, or air-flow velocity during dispensing through the disposable cartridge.

In some examples, the method performed includes, upon reaching a predefined end-of-life criterion, writing, using at least the communication interface system, an irreversible usage flag to inhibit further dispensing by the disposable cartridge.

In some examples, the method performed includes disabling operation or activation of the electrostatic applicator upon detection of an irreversible usage flag.

In some examples, the method performed includes adjusting the configured operating parameters during dispensing in response to environmental measurements of one or more sensors of the disposable cartridge and/or the electrostatic applicator.

In some examples, the method performed includes recording an event log that includes a cartridge identifier, environmental conditions, and parameter adjustments, the event log being cryptographically signed and stored in secure memory.

In some examples, the method performed includes wirelessly communicating, using at least the NFC integrated circuit of an NFC tag of the communication interface system, cartridge operational information to a graphical user interface.

In some examples, the method performed includes selecting, in response to data received from the NFC integrated circuit, a nozzle geometry from a library of nozzle geometries for differing profiles of treatment solutions.

In some examples, the method performed includes producing, using at least a nozzle assembly of the disposable cartridge, an electrostatically charged spray plume having a predetermined average droplet diameter and/or charge-to-mass ratio established by the configured spray-generation and electrostatic-charging parameters.

In some examples, configuring the operating parameters includes setting a voltage level that is modulated according to a flow rate commanded by the at least one spray-generation parameter.

In some examples, a disposable fluid delivery system includes a disposable cartridge containing and/or configured to be filled with or otherwise receive a treatment solution and a nozzle assembly configured to dispense the treatment solution. A communication interface system can be coupled with the disposable cartridge. The communication interface system can include one or more communication or identification modules and a memory storing an encrypted data set that includes operating parameters that include at least one of a spray-generation parameter, an electrostatic-charging parameter, and an authenticity credential. The memory can include executable instructions that, when executed by one or more processors, cause the communication interface system to exchange data with an electrostatic spray-generation subsystem, retrieve and decrypt the encrypted data set, authenticate the disposable cartridge based on the authenticity credential, and configure the electrostatic spray-generation subsystem in accordance with the operating parameters obtained from the decrypted data set.

In some examples, the communication module includes at least one of near-field communication (NFC), radio-frequency identification (RFID), optical code readers including QR code readers, Bluetooth Low Energy (BLE), Wi-Fi, or other wireless or optical communication technologies.

In some examples, the disposable cartridge is configured to receive a syringe containing the treatment solution.

In some examples, the disposable cartridge is removably attachable to a housing of the electrostatic applicator.

In some examples, the disposable cartridge includes a fluid reservoir containing a treatment solution.

In some examples, the disposable cartridge includes memory storing information associated with operational parameters of the treatment solution.

In some examples, the cartridge or a syringe assembled with the cartridge is configured to be filled with the treatment solution.

In some examples, the instructions cause the communication interface system to write, after a predetermined eject-mass or elapsed-time threshold is reached, a non-reversible usage flag to disable subsequent reuse of the cartridge.

In some examples, the communication interface system is a one-time-programmable or write-once, read-many (WORM) device that prevents alteration of the encrypted data set.

In some examples, the spray-generation parameter includes at least one of flow rate, pulse width, pulse frequency, and fluidic pressure set-point.

In some examples, the electrostatic-charging parameter includes at least one of applied voltage magnitude, current limit, discharge waveform, and polarity.

In some examples, the instructions cause the communication interface system to select from a library of nozzle geometries optimized for different rheological profiles of one or more treatment solutions.

In some examples, the instructions cause the communication interface system to store nozzle-specific and material-specific parameters so that spray performance is tuned for a dispensing combination of nozzle and formulation parameters.

In some examples, the system further includes a plurality of sensors configured to measure operating parameters of the cartridge including at least one of ambient humidity, motor speed, applied voltage, intake of air, ambient temperature, and air-flow velocity, and the instructions cause the communication interface to dynamically adjust at least one of the spray-generation and electrostatic-charging parameters based on sensor feedback.

In some examples, the system further includes a plurality of sensors configured to measure operating parameters of the cartridge including at least one of ambient humidity, motor speed, applied voltage, intake of air, ambient temperature, and air-flow velocity, and the instructions cause the communication interface to dynamically adjust at least one of motor speed, intake of air, and applied voltage based on retrieved information.

In some examples, the instructions cause the communication interface system to log cartridge identification, usage metrics, and environmental data to a tamper-resistant memory for post-procedural traceability.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A depicts a perspective view of an example electrostatic applicator positioned in an exploded state with an example base.

FIG. 1B depicts a side view of the example electrostatic applicator of FIG. 1A positioned in an exploded state with the example base.

FIG. 1C depicts a side view of the example electrostatic applicator of FIG. 1A positioned in an assembled state with the example base.

FIG. 2A depicts a perspective view of the example electrostatic applicator of FIGS. 1A-1C with a disposable cartridge in an exploded state.

FIG. 2B depicts a perspective view of the example electrostatic applicator of FIGS. 1A-1C with the disposable cartridge of FIG. 2A in the exploded state.

FIG. 3 depicts a side view of the example electrostatic applicator of FIG. 1A positioned with a portion of an outer housing removed.

FIG. 4A depicts a forward perspective view of the example cartridge of FIGS. 1A to 2B.

FIG. 4B depicts a rear perspective view of the example cartridge of FIGS. 1A to 2B.

FIG. 5A depicts a lower rear perspective view of the example cartridge of FIGS. 1A to 2B.

FIG. 5B depicts a side view of the example cartridge of FIGS. 1A to 2B with a portion of its outer housing removed.

FIG. 5C depicts an exploded perspective view of internal components of example cartridge of FIGS. 1A to 2B with its outer housing removed.

FIG. 6 depicts a close-up cross-section view of section 6 from FIG. 3 showing aspects of the example cartridge of FIGS. 1A to 3 in a closed position with the electrostatic applicator.

FIG. 7A depicts a close-up cross-section view of example aspects of FIG. 3 showing a closed system in the air intake and exhaust ports with respect to the example electrostatic applicator and the example cartridge.

FIG. 7B depicts a close-up cross-section view similar to FIG. 7A but with an example valve seal in an open position.

FIG. 8A depicts a side cross-section view of example aspects of the actuator previously shown in FIGS. 1A to 3.

FIG. 8B depicts a side cross-section view of another example actuator previously contemplated for use with the examples of FIGS. 1A to 3.

FIG. 9A depicts a forward perspective view of an example electrospinning cartridge contemplated for use with the example electrostatic applicator of FIGS. 1A to 3.

FIG. 9B depicts a rear perspective view of the example electrospinning cartridge contemplated for use with the example electrostatic applicator of FIGS. 1A to 3.

FIG. 10A depicts a lower rear perspective view of the example cartridge of FIGS. 9A-9B.

FIG. 10B depicts a side view of the example cartridge of FIGS. 9A-9B with a portion of its outer housing removed.

FIG. 10C depicts an exploded perspective view of internal components of example cartridge of FIGS. 9A-9B with its outer housing removed.

FIG. 11A depicts a perspective view of internal components of an example electrostatic cartridge contemplated for use with the example electrostatic applicator of FIGS. 1A to 3, the components including a pair of syringe and electrostatic nozzles.

FIG. 11B depicts a side cross-section view of the example internal components shown in FIG. 11A.

FIG. 12A depicts a perspective view of internal components of an example electrostatic cartridge contemplated for use with the example electrostatic applicator of FIGS. 1A to 3, the components including a pair of syringes, at least one electrostatic nozzle, and at least one electrospinning delivery tube.

FIG. 12B depicts a side cross-section view of the example internal components shown in FIG. 12A.

FIG. 13A depicts a perspective view of internal components of an example electrostatic cartridge contemplated for use with the example electrostatic applicator of FIGS. 1A to 3, the components including a pair of syringes and electrospinning delivery tubes interconnected by a Y-shaped member.

FIG. 13B depicts a side cross-section view of the example internal components shown in FIG. 13A.

FIG. 14A depicts a side perspective view of an example cone configured for use with any of the herein disclosed nozzles of the described disposable cartridges.

FIG. 14B depicts a side perspective view of another example cone configured for use with any of the herein disclosed nozzles of the described disposable cartridges.

FIG. 14C depicts a side perspective view of another example cone configured for use with any of the herein disclosed nozzles of the described disposable cartridges.

FIG. 14D depicts a side perspective view of another example cone configured for use with any of the herein disclosed nozzles of the described disposable cartridges.

FIG. 15 is a component diagram of various components and systems that can be included with electrostatic and/or electrospinning devices, according to the present disclosure.

FIG. 16 is component diagram of the user interface of FIG. 15, according to the present disclosure.

FIG. 17 is a component diagram of the controller of FIG. 15, according to the present disclosure.

FIG. 18 is a flow diagram of example processes for operating the electrostatic applicator and disposable cartridge system, according to the present disclosure.

FIG. 19 is a flow diagram of example processes for operating the electrostatic applicator and disposable cartridge system, according to the present disclosure.

FIG. 20 is a flow diagram of example processes for operating the electrostatic applicator and disposable cartridge system, according to the present disclosure.

FIG. 21 is a flow diagram of example processes for operating the electrostatic applicator and disposable cartridge system, according to the present disclosure.

FIG. 22 is a flow diagram of example processes for operating the electrostatic applicator and disposable cartridge system with interactive components that provide feedback to a user regarding the status of the device, according to the present disclosure.

FIG. 23 is a flow diagram of example processes for operating the electrostatic applicator and disposable cartridge system with interactive components that provide feedback to a user regarding the status of the device, according to the present disclosure.

FIG. 24 is a flow diagram of example processes for operating the electrostatic applicator and disposable cartridge system with interactive components that provide feedback to a user regarding the status of the device, according to the present disclosure.

FIG. 25 is a flow diagram of example processes for operating the electrostatic applicator and disposable cartridge system with interactive components that provide feedback to a user regarding the status of the device, according to the present disclosure.

FIG. 26 is a flow diagram of example processes for operating the electrostatic applicator and disposable cartridge system with interactive components that provide feedback to a user regarding the status of the device, according to the present disclosure.

FIG. 27 is a flow diagram of example processes for operating the electrostatic applicator and disposable cartridge system with interactive components that provide feedback to a user regarding the status of the device, according to the present disclosure.

FIG. 28 is a flow diagram for using a proximity sensor to provide feedback to a user regarding optimal applicator and disposable cartridge system positioning, according to the present disclosure.

FIG. 29 is a flow diagram for operating an example applicator and disposable cartridge system, according to the present disclosure.

FIG. 30 is a flow diagram for operating an example applicator and disposable cartridge system, according to the present disclosure.

FIG. 31 depicts an illustration of system according to an example embodiment of this disclosure.

FIG. 32A is an example user interface for operating an example user device according to the present disclosure.

FIG. 32B is an example user interface for operating an example user device according to the present disclosure.

FIG. 33 is a flow diagram for operating an example electrostatic applicator system according to the present disclosure.

FIG. 34 is a flow diagram for a computer-implemented method for operating any electrostatic applicator system of this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In this disclosure, relative terms, such as “about,” “substantially,” or “approximately” are used to indicate a possible variation of ±10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a treatment site of a “subject” or “patient” may be a wound site or treatment of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As used herein, the term “operator” refers to, and is not limited to, a doctor, surgeon, nurse, physical therapist, or any other healthcare professional, as well as any suitable individual or automated system configured to apply or facilitate the application of a treatment solution to a treatment site of a subject. The term “operator” may further encompass delivery instrumentation or devices associated with the administration or control of the treatment process.

As used herein, the term “treatment solution” refers to one or more fluids (e.g., liquids, emulsions, gels, and/or mixtures thereof) that may include, without limitation, an antiseptic solution, disinfectant solution, analgesic, exosome preparation, biologic, chlorohexidine gluconate, povidone-iodine, and/or a liquid bandage formulation. The treatment solution may be provided as a single composition or as a combination of multiple components. In certain embodiments, the analgesic component may include one or more of lidocaine, levobupivacaine, acemetacin, ketorolac, and/or ceftazidime. The biologic component may include one or more of stem cells and/or mammalian primary cells, medicaments, gels (e.g., hydrogels), or reconstitutable materials (e.g., immiscible or lyophilized ingredients mixable with one or more solvents prior to administration). The disinfectant component may include one or more alcohols, aldehydes, oxidizing agents, phenolic compounds, quaternary ammonium compounds, antibacterial agents, biguanides, surfactants, analgesic agents, debridement agents, or any other medically suitable content or medicament configured for storage within a cartridge and for subsequent delivery (e.g., application, deposition, and/or electrostatic spraying) to a treatment site of a patient. The treatment solution may include any concentration, ratio, or combination of the aforementioned components.

In certain embodiments, the term “treatment solution” may further encompass one or more tracking materials (e.g., gels or compositions including traceable or imaging-compatible constituents intermixed with the treatment solution). The treatment solution may include any number of small-molecule drugs, peptides, cells, or other therapeutic agents. In some aspects, the treatment solution may further include one or more active pharmaceutical ingredients, growth factors, trophic factors, exosomes, mammalian regenerative cells, and/or a supportive or carrier matrix. In some aspects, the treatment solution may include one or more analgesics such as lidocaine, levobupivacaine, acemetacin, ketorolac, or combinations thereof.

As used herein, the terms “distal” and “proximal” are employed to describe relative positions or directions with respect to a designated reference point (e.g., a user such as a treating physician or medical interventionalist). The term “distal” (or “distally”) refers to a position or direction that is farther from the reference point. Conversely, the term “proximal”, “proximally”, or “proximate” refers to a position or direction that is nearer to the reference point.

As used herein, a “communication interface system” can refer to circuitry, firmware, software, and associated components that enable electronic identification, data exchange, and control signaling between the components, such as any of the herein disclosed cartridges and one or more external devices, including but not limited to an electrostatic applicator and/or a user device. In some examples, the communication interface system can include a communication module and a memory subsystem, where the memory subsystem store information (e.g., cartridge-specific information) and executable instructions, which, when executed by one or more processors, facilitate secure data transactions such as authentication, retrieval and decryption of operating parameters, configuration of applicator subsystems, logging of usage and environmental data, and enforcement of usage constraints.

In some examples, the communication interface system includes or is implemented as a near-field communication (NFC) integrated circuit positioned with the cartridge. The NFC integrated circuit can include an antenna and memory configured to wirelessly exchange data with a complementary reader. In other examples, or in combination with NFC, the communication module can include at least one of radio-frequency identification (RFID), optical code readers (e.g., QR code readers), Bluetooth Low Energy (BLE), Wi-Fi, or other wireless or optical communication technologies suitable for short- or medium-range data transfer. The communication interface system can be configured for unidirectional or bidirectional data exchange, can operate in a read-only, write-once, or read-write mode, and can include cryptographic capabilities for encrypting, decrypting, and digitally signing data to authenticate the cartridge and protect the integrity of operating parameters.

An increasing amount of research, development, and clinical attention has been directed toward improving the efficacy and precision of pain management and infection control therapies. Conventional approaches such as oral or intravenous (IV) drug administration remain the most prevalent treatment methods. However, these systemic delivery routes often exhibit limited therapeutic efficiency, as the active agents are distributed throughout the entire body rather than being localized to the treatment site. Consequently, the therapeutic effect may be diluted, and unintended systemic side effects may arise.

To address these limitations, various localized delivery modalities have been developed, including topical formulations and hydrogel-based carriers, which allow for spatial and temporal control of therapeutic release. These hydrogel and polymeric carriers enable localized application of small molecules, peptides, biologics, and cellular therapies. Nonetheless, such materials can present drawbacks. For example, polymeric carriers may be fragile, difficult to sterilize, and costly to manufacture, thereby limiting their practical use in acute or field-based medical settings.

In recent years, electrostatic or electrospray delivery techniques have been explored as promising alternatives for localized administration of treatment solutions such as antibacterial, antiseptic, and analgesic compositions. Electrostatic spraying involves subjecting a treatment solution to an electric field that imparts a charge to the fluid. The charged droplets can then be directed toward a treatment site with controlled precision. In biomedical applications, this process can be particularly advantageous since the resting potential of human tissue and cellular membranes is generally negative. The resulting electrostatic attraction between the positively charged treatment solution and the negatively charged tissue surface enhances deposition efficiency and treatment localization.

Beyond electrostatic attraction, electrospraying also provides additional benefits, such as producing micron or submicron sized droplets that create a substantially uniform coating over the target area. When the electrical stress on a charged droplet exceeds the cohesive surface tension of the liquid, the droplet undergoes Rayleigh disintegration, also referred to as Coulomb fission, thereby atomizing into smaller droplets. As used herein, the term “atomize” refers to converting a liquid treatment solution into fine droplets or particles. Parameters such as the solvent dielectric constant, electrical conductivity, vapor pressure, viscosity, and miscibility, as well as the applied voltage, can influence the droplet morphology, charge distribution, and overall atomization dynamics.

Prior electrostatic spraying devices were not designed to account for these interdependent parameters. Traditional applications such as inkjet printers and paint sprayers operated with uniform solutions, fixed flow rates, and standardized voltage potentials, offering little flexibility for varying fluid properties or biomedical use cases. Moreover, such systems did not address sterility, single use safety, or adaptive control for medical grade treatments.

The present disclosure provides communication enabled disposable cartridges and applicators, for example NFC enabled cartridges, that are prefilled, sterile, and configured to store and/or otherwise receive (e.g., in a preloaded or loadable syringe) one or more treatment solutions suitable for electrostatic application. Each disposable cartridge can communicate with a reusable electrostatic applicator or control unit to exchange identifying and operational data such as solution composition, viscosity, conductivity, and recommended spraying parameters including voltage, droplet size, and flow rate. This communication enables automatic calibration of the applicator for optimized atomization and delivery tailored to the specific treatment solution.

In certain embodiments, embedded memory or circuitry can be included with the cartridge and/or applicator configured to transmit data wirelessly, for example using NFC, RFID, or Bluetooth protocols. Such connectivity ensures traceability, dosage control, and sterility verification for each administration. Accordingly, the system provides a safe, efficient, and personalized delivery mechanism capable of generating nano to microscale droplets for targeted, site specific therapy.

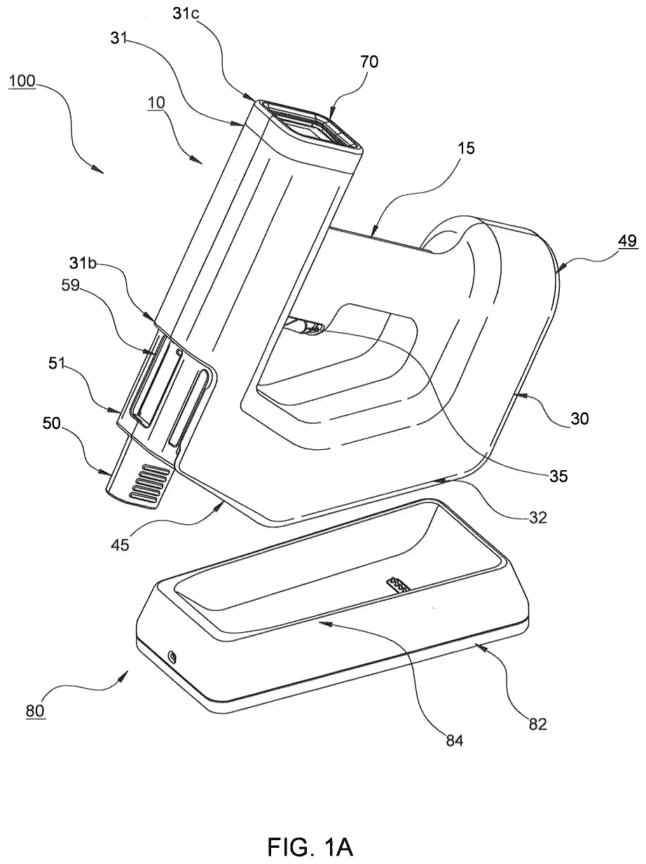

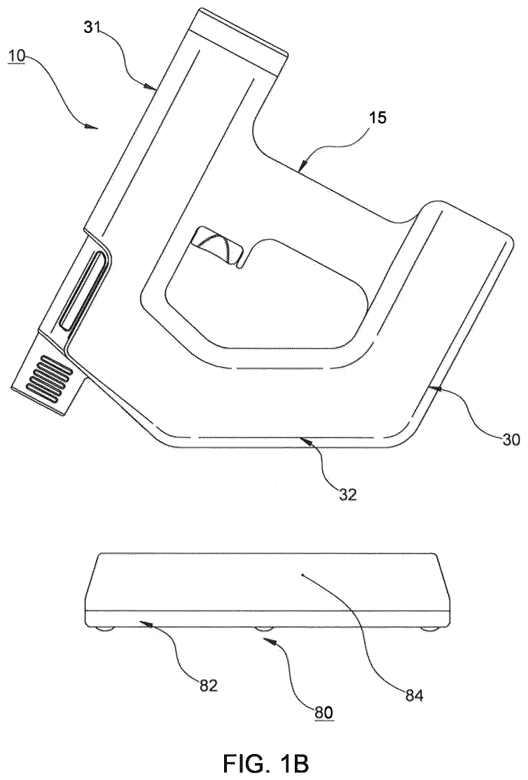

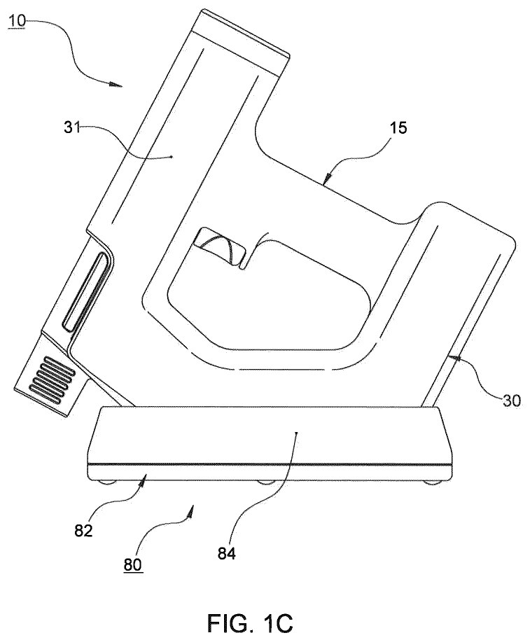

Turning to the drawings, FIG. 1A provides an example applicator 100 positioned in an exploded state with an example base 80. Applicator 100 can be used in conjunction with a disposable cartridge 50, according to the present disclosure. Applicator 100 can include an applicator housing 10 which can include an upper portion 31, a lower base portion 30 at the lower section, and a handle portion 15 between portions 30 and 31. A forward cartridge support portion 32 can also be positioned between portions 30 and 32. In some aspects, an obtuse angle can be formed between aspects of outer surfaces of portions 30 and 32 while handle portion 15 can be orthogonal to portions 30 and/or 31. And while shown as a handheld pistol shaped device, nothing requires the reusable applicator 100 to have a pistol-shaped design, as the components herein can also be combined in other electrosprayer designs, for example and not limitation a fully-cylindrical, handheld electrosprayer design.

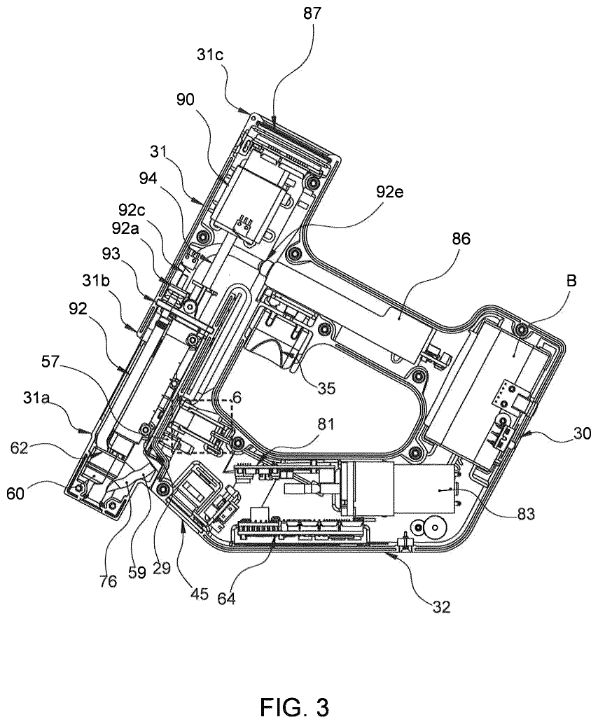

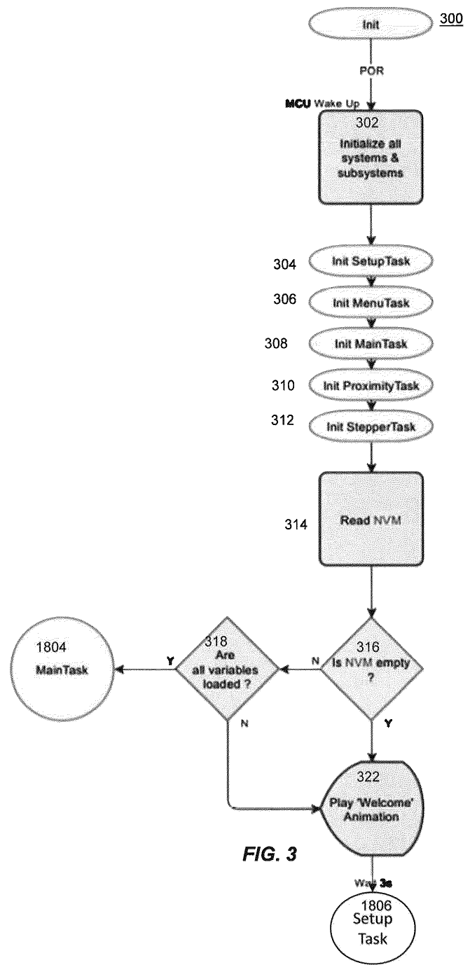

As shown more clearly in FIG. 3, portion 30 can house a battery B that provides the voltage potential to create the electrical field at the nozzle assembly 60 of cartridge 50 and/or can power the components of applicator 100 (e.g., CPU and/or HV module 86). Battery B can include one or more batteries, including for example direct current batteries such as lithium ion batteries. Battery B can provide sufficient voltage to create the voltage potential described above, including but not limited to approximately 1 V to approximately 40 kV. In some examples, the range can be approximately 1 V to 8 kV. In some examples, the voltage supply of battery B can be one or more rechargeable batteries. In this example, aspects of the applicator 100, such as portion 32, can engage with the charging base 80 that can in turn charge the voltage supply of battery B (e.g., inductively) when the device is not in use. Portion 30 can be configured to be detached from the charging base 80 by pressing an electrostatic applicator release (not shown). In some examples, applicator 100 can include an actuator, e.g., a button 35 that can activate and/or initiate the components of applicator 100. For example, the button 35 can initiate power from battery B (see FIG. 3), which can include a rechargeable module battery such as a direct current battery. By activating and/or initiating power from battery B, the system is powered to electrostatically charge the liquid solution of the cartridge via direct charging, induction charging, indirect charging, or any combinations thereof. In the case of direct charging, liquid solution of the cartridge can flow through an electrically conductive tube or other conduit that is electrostatically charged such that the liquid solution is contacted and charged by direct contact.

In some examples, battery B can power the high voltage (HV) module 86, air pump 83, circuit board 64, target sensor 45, motor 90, user interface 87, one or more processors of applicator 100 (e.g., central processing unit (CPU)) as well as components of cartridge 50. As will be described in greater detail below, other features can be included in applicator 100 that can obviate the need for button 35, including for example accelerometers, activation inputs from a user device, etc.

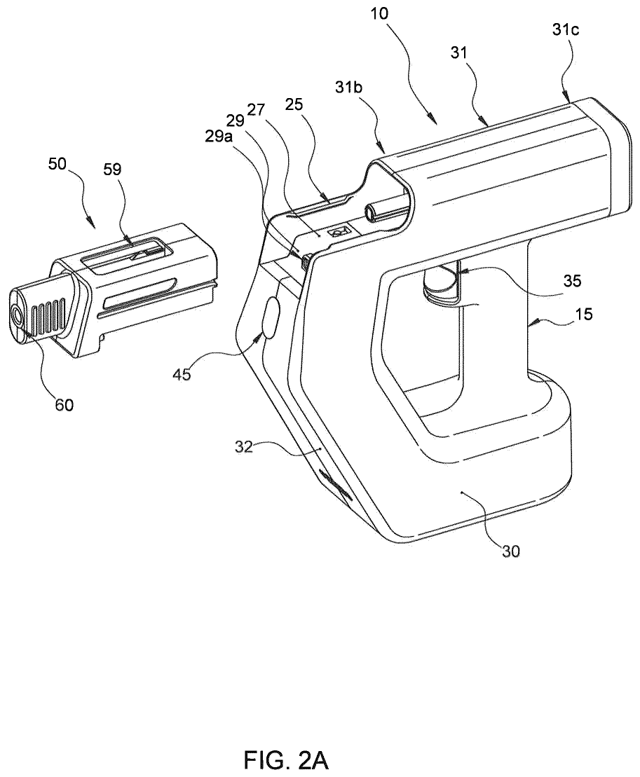

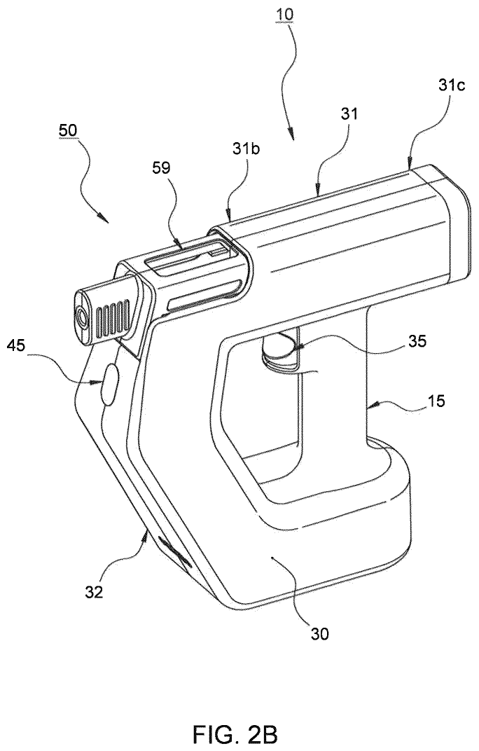

Applicator housing 10 can include a cartridge chamber 27 sized and positioned to accept the housing 49 of the disposable cartridge 50, as explained more particularly below (see FIGS. 2A, 2B, 3, 4A, and 4B). Cartridge chamber 27 can include a distal stepped portion 29 through which connecting ports of internal components of housing 10 can connect to aspects of cartridge 50 (e.g., air supply ports). Although the examples shown in FIGS. 1A-1C position the cartridge chamber 27 at the top of a gun-shaped, portable, cordless, reusable applicator 100, nothing requires to the cartridge chamber 402 to be so located. For example, the cartridge chamber 27 can be positioned on the side of applicator 100 (e.g., the side of the housing 10 of applicator 100, or any other location). In some aspects, HV module 86 can include electrical components for powering the components used for charging and spraying the liquid solution of cartridge 50. For example, HV module 86 can include the components used for providing electricity from voltage wire 92c through port 92a of cartridge 50 and to wire 92d so as to control the voltage applied to the liquid solution of cartridge 50, and/or for providing electricity to actuate plunger 71 (or motor 90 for operating syringe 70 including advancing plunger 71) so as to control the flow rate of liquid solution from cartridge 50 and through nozzle assembly 60.

In some aspects, HV module 86 can be configured to adjust or otherwise control operational aspects of cartridge 50 including but not limited to frequency, duty cycle, and input voltage to yield varying output voltages at different efficiencies. In some aspects, HV module 86 can be a closed loop system that monitors the output voltage and adjusts input parameters to optimize the output to a desired voltage for use with cartridge 50. HV module 86 can also be configured to produce positive and/or negative high voltage using the same board. In some aspects, HV module 86 can include a printed circuit board that is double sided so as to minimize footprint used within housing 10. In some aspects, HV module 86 can include a dedicated module to generate positive high voltage. In some aspects, HV module 86 includes a plurality of diodes configured to be physically rotated (e.g., rotated approximately 180°). In some aspects, the diodes can be configured to be rotated or otherwise adjust their orientation based on operational instructions of the system. In so rotating, in this example HV module 86 is configured to flip a polarity of its diodes in the multiplier stages so that HV module 86 is able to generate positive high voltage and negative high voltage depending on the orientation of the rotatable diodes. In some aspects, HV module 86 can include a first positive high voltage multiplier system and a second negative high voltage multiplier system physically separate from the first positive high voltage multiplier system. Each of the subsystems of the HV module 86 can be positioned on the same circuit board and can be selectively actuatable during applicator 100 operations, as needed or required. In some aspects, each of the subsystems can be positioned on separate circuit boards.

Applicator 100 can include one or more processors, for example CPU, that can facilitate activation of applicator 100, receiving and outputting signals relating to the voltage, flow rate, proximity, etc. for the particular liquid treatment solution, and the like.

Within housing 10, applicator 100 can include a piston 94 and corresponding motor 90 that actuates syringe 70. Piston 94 can be positioned between motor 90 and HV wall 93. On the opposite side of HV wall 93, cartridge 50 can be positioned when attached to chamber 27 of housing 10. In some aspects, when cartridge 50 is positioned as in FIG. 3, a plunger, piston, or other aspects within a barrel of syringe 70 of cartridge 50 can be advanced by piston 94, through HV wall 93, to advance contents of cartridge 50 through nozzle assembly 60. In some aspects, motor 90 can be positioned within portion 31 adjacent a user interface 87. In some aspects, motor 90 can be a stepper motor, a worm drive motor, solenoid, and the like. The speed of motor 90 can be adjusted based on aspects such as preferred flow rate for the particular liquid solution being emitted by nozzle assembly 60 of cartridge 50.

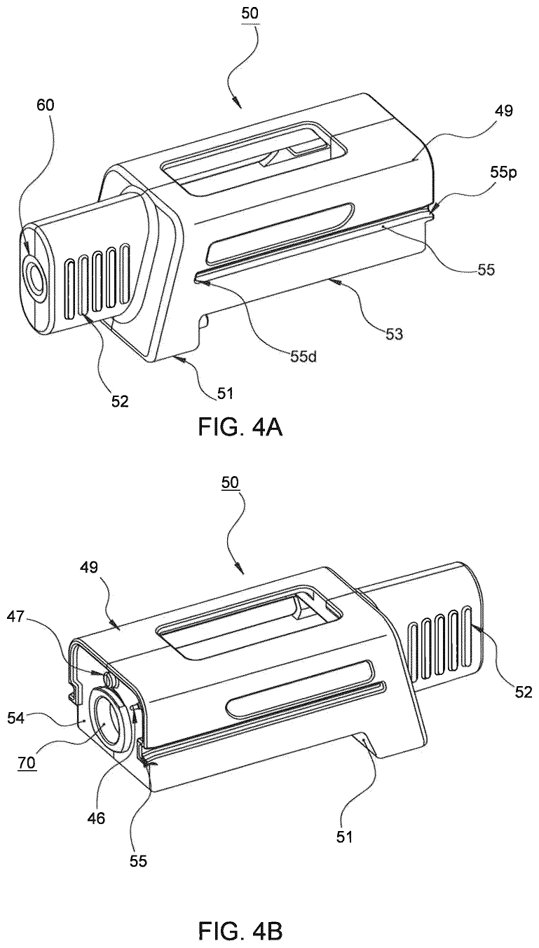

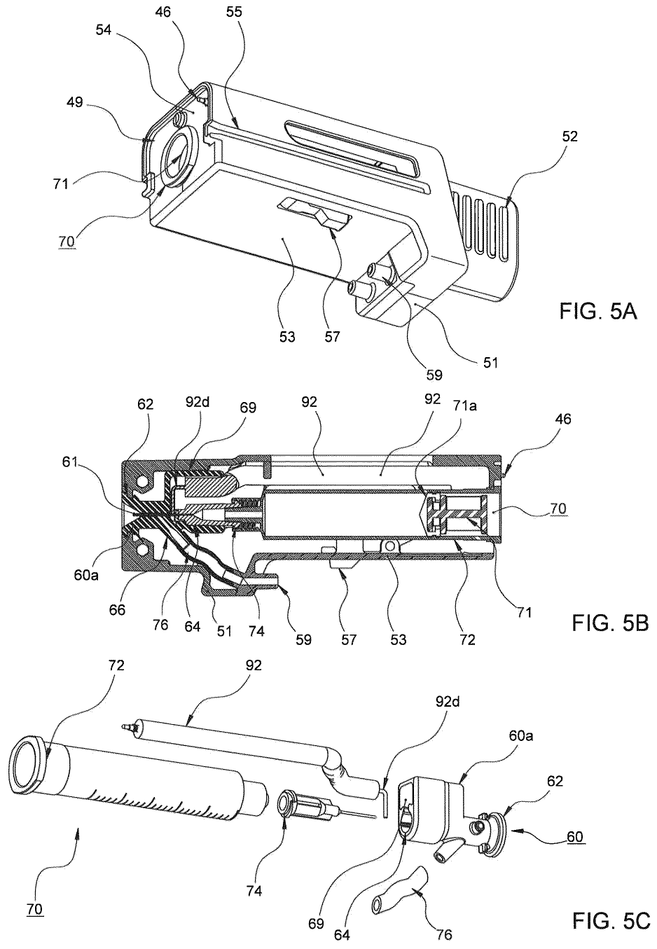

As shown in FIG. 3, housing 10 can include a voltage wire 92c that contacts the voltage contact 92e of HV module 86 and cartridge voltage contact 92a of HV wall 93. Cartridge 50 electrically connects to voltage contact 92a, via cartridge HV contact 46 (see FIGS. 4B, 5A, 5B) when assembled in chamber 27 of housing 10. In operation, wire 92c provides an electrical connection between the voltage supply of high voltage module 86, which is powered by battery B. Within portion 32 of housing 20 (e.g., underneath chamber 27), an air pump 83 connects with a supply end of air supply tube 81. When cartridge 50 is assembled with chamber 27, air supply tube 81 in turn supplies air to air supply tube 76 of cartridge 50 so as to provide air for spraying charged liquid droplets. The air supply of air pump 83 can provide the high velocity air supply without the need for a separate air hose. In other embodiments, housing 10 can be connected to an external air hose that provides an air supply for spraying.

Applicator 100 can include a display screen with its user interface 87, which can include a liquid crystal (LCD) and/or a light emitting diode (LED) display. The display screen of user interface 87 can enable an operator of applicator 100 to receive information relating to the status of applicator 100, including operational parameters of cartridge 50. For example, the display screen of user interface 87 can display information related to what type of liquid solution is within disposable cartridge 50 that is connected to applicator 100. As described above, this information can be written on integrated memory of disposable cartridge 50, and a CPU of applicator 100 can receive this information and display the information to the operator of applicator 100. In some aspects, user interface 87 can display welcome animations, display whether the electrostatic applicator has connected with a user device (as will be described below), display whether applicator 100 has been properly grounded, display whether a disposable cartridge 50 has been loaded into cartridge chamber 27, and display what parameters are being used for the particular liquid solution (e.g., flow rate, voltage supply, droplet size, intended target, intended patient condition, recommended proximity, etc.).

In some examples, the display screen of user interface 87 can also have touchscreen capabilities. For example, user interface 87 can act as an actuator to initiate or otherwise control the voltage supply to the voltage wire 92d of cartridge 50, initiate or otherwise control air flow into air supply tube 76, and/or initiate or otherwise control the plunger 71 of syringe 70 to expel fluid from syringe 70 through nozzle assembly 60. As will be described in greater detail below, other initiation mechanisms can be used to spray the fluid, including an actuator 35 (e.g., a mechanical trigger, a switch, an actuator, and/or a graphical user interface configured to receive input from a user and perform one or more related operations) and/or signals from an external user device.

Referring to FIG. 4A, a forward perspective view of cartridge 50 is provided while FIG. 4B depicts a rear perspective view of cartridge 50. Similarly, FIG. 5A depicts a lower rear perspective view of cartridge 50. FIG. 5B depicts a side view of cartridge with a portion of its outer housing 49 removed while FIG. 5C depicts an exploded perspective view of internal components of cartridge 50 with outer housing 49 removed. FIG. 5B also shows a detailed view of the nozzle housing 60a. It is understood that in some aspects, the entire housing 60a can be molded (e.g., insert molded) as a single, integrally formed part and/or formed of multiple parts or sections. The nozzle housing 60a can include a nozzle outlet channel 62 on a distal end that can be funnel shaped, sized and positioned to contain the outlet end of delivery tube 61. The nozzle housing 60a can include an air inlet port 66 in fluid communication with the nozzle end of the air supply tube 76 and inlet port 59 of cartridge 50.

The nozzle housing 60a can include a voltage cavity port 69 for connecting the voltage wire 92d and corresponding voltage tube 92 to delivery tube 61. One side of the port 69 (e.g., a distalmost end of the port 69) can include a contact section through which the wire 92d can pass to contact delivery tube 61. At another end of the port 69 (e.g., a proximal end of the port 69), the cavity associated with port 69 can be tubular for securing the outer surface of the voltage tube 92 in a friction fit. The nozzle outlet channel of housing 60a described above can be positioned at a distal end of housing 60a and can receive air from port 66 and fluid delivery tube 61 and expel fluid droplets that are charged by the voltage wire 92d. In some aspects, wire 92d can electrostatically charge contents within delivery tube 61 as well as fluid contents proximal thereof (e.g., contents within barrel portion 72 of syringe 70). As shown in FIGS. 5B and 5C, the voltage wire 92d can include one or more curved surfaces (e.g., at least one angled down curve to connect through tube 92, port 69 and in contact with delivery tube 61). Wire 92d can include any number of shapes, including but not limited to an S-or serpentine shape. To facilitate the shape of wire 92d, tube 92 can include one or more curves or bends, as shown.

Housing 49 can be formed of a multi-part shell with an aligning groove 55 that can engage with the aligning tab 25 of cartridge chamber 27. Housing 49 can be formed and/or assembled in a number of ways, including but not limited to, machining, molding, injection molding, three-dimensional printing, or any other suitable manufacturing process. Suitable materials for housing 49 may include one or more of glass filled nylon, glass filled polypropylene, glass filled polyethylene, polypropylene, polyethylene, or a plastic material. In some examples, housing 49 can include two or more sections of moldable plastic, e.g., a first half and a second half. The sections of housing 49 can be assembled together with fasteners (e.g., screws, rivets, a weld [e.g., a sonic weld], one or more straps or snaps, an adhesive or adhesive tape, etc.) such that the internal components are disposed between the portions and/or respective halves of housing 49. In some aspects, housing 49 can include a housing spray outlet associated with nozzle assembly 60 that enables charged treatment solution to be expelled from cartridge 50.

Groove 55 can engage with tab 25 to align housing into the cartridge chamber 27 with proper alignment between cartridge 50 and chamber 27. Housing 49 can include one groove 55 positioned on opposite lateral sides of cartridge 50. Groove 55 can include an open proximal end 55p and a closed distal end 55d. When end 55d of groove 55 is adjacent or otherwise towards nozzle assembly 60, end 55d prevents tab 25 from sliding deeper into groove 55. In some aspects, proximal end 55p can be opposite end 55d and may include an open, funnel or tapered shape to facilitate aligning and engagement between groove 55 and tab 25.

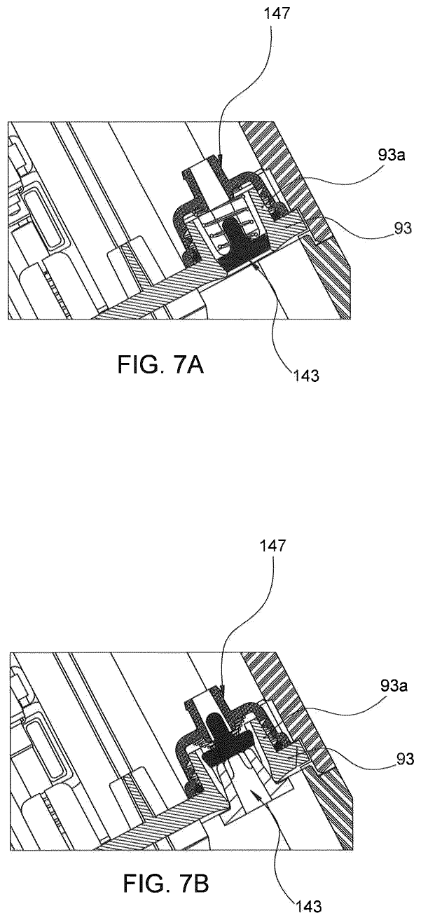

FIG. 4B specifically shows the supply end and contact end of cartridge 50. HV contact 46 can be a contact port embedded on HV wall 54 and/or otherwise positioned on a face of cartridge HV wall 54, which is configured to mount against HV wall 93 of housing 10. HV wall 54 can also include an air intake port 47 which can be configured to couple to a corresponding air intake port 147 of HV wall 93 (see, e.g., FIGS. 7A and 7B). As shown in FIG. 3 and FIG. 5A, cartridge 50 may also include air supply ports 59 configured to receive air supply tube 81 to place tubes 81, 76 and pump 83 in fluid communication with each other. Air supply port 59 may be positioned on the stepped portion of housing 49 which is shaped to correspondingly attach to stepped portion 29 of housing 49. The supply port 59 of the air supply tube 81 can extend at least partially from the housing 49 and be shaped to insert into an air supply port 29a on of applicator housing 10 at or adjacent stepped portion 29. In some aspects, air supply tube 76 of cartridge 50 and air supply tube 81 of housing 10 can include a combination of both flexible and rigid materials (e.g., nylon braiding). In some aspects, air supply tube 76 of cartridge 50 and air supply tube 81 of housing 10 can be manufactured from a pliable, fluid tight material, such as an elastomer, rubber, silicon, polyvinyl chloride, etc. In some aspects, air supply tube 76 of cartridge 50 and air supply tube 81 of housing 10 can also be can be insert-molded and/or overmolded with a combination of softer, pliable fluid tight materials and/or one or more rigid materials (e.g., metal, alloy, etc.).

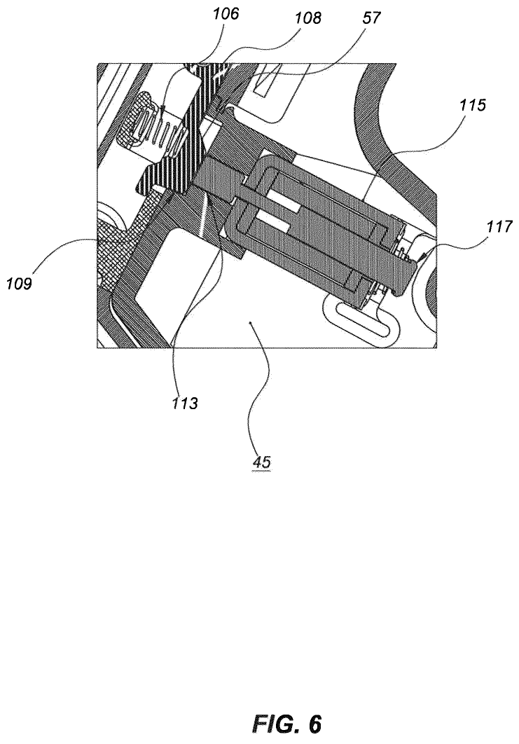

Once securely engaged with chamber 27, cartridge 50 can be released by a release button 57. As shown in FIGS. 5A, 5B, and FIG. 6, button 57 can extend at least partially though a lower surface 53 of housing 49 via an aperture in the lower surface 53 of housing 49. Button 57 can be a latch that is connected, for example, to a portion of the syringe 70 and can be removed from cartridge 50 when cartridge 50 is ready to be attached to housing 10. This can indicate to a user that the cartridge 50 has not yet been used and that the contents therein (e.g., liquid treatment solution) has not been used. In some examples, the contents stored within syringe 70 can be sterilized, and release button 57 can indicate that the contents of disposable cartridge 50 are sterile and have not been used (e.g., not yet used on a previous patient). In some aspects, button 57 can prevent syringe 70 and any components thereof from advancing contents therein prior to use. In some aspects, button 57 can a include near field communication (NFC) tag with identifying cartridge information and other operational parameters readable therefrom indicate to a user that cartridge 50 has not yet been used and that the contents therein (e.g., liquid treatment solution) has not been used.

As also shown in FIGS. 5A, 5B, and FIG. 6, cartridge 50 can contain and/or at least partially enclose, within its housing 49, the aforementioned air supply tube 76, voltage tube 92, and syringe 70. Syringe 70 can be an assembly that includes a glass or plastic syringe for storing the liquid solution that is to be applied using the disposable cartridge 50. Syringe 70 can be assembled within the disposable cartridge 50 pre-filled with the desired liquid solution. At a proximal end, syringe 70 can include movable plunger 71, a moveable stopper 71a, and a barrel portion 72 extended distally therefrom. Plunger 71 and stopper 71a are configured to advance within barrel 72 so as to advance contents of barrel portion 72 through a luer lock 74 positioned at distal end of portion 72. An inner distal delivery tube 61 can be distally extended from luer lock 74 through nozzle assembly 60. Delivery tube 61 can be substantially tubular with a blunt distal tip. However, delivery tube 61 is not so limited and may include a puncture tip. Delivery tube 61 can preferably range between approximately 15 gauge (outer diameter of .072 inches and inner diameter .054 inches) to approximately 30 gauge (outer diameter of .01225 inches and inner diameter of .00625 inches). At a distal end of luer lock 74, a distal end of voltage wire 92d can run through nozzle tube 92 and be connected to a proximal portion of inner distal delivery tube 61, which can be constructed from one or more conductive materials. Wire 92d can include a needle-like structure made of one or more conductive materials (e.g., metal) that can create the electric field to charge the liquid solution that flows from syringe 70 through nozzle assembly 60. In operation, syringe 70 can be configured to deliver the stored liquid solution at a predetermined rate from barrel portion 72 through delivery tube 61 and ultimately sprayed via nozzle assembly 60 onto a treatment site (e.g., a wound site of a patient).

In some aspects, voltage wire 92d can be in electrical communication with a voltage source at one end (e.g., port 46) and a nozzle tube (e.g., nozzle tube 92d) at a nozzle end as shown in FIGS. 5B and 5C. While not shown, it is contemplated that in some aspects contact 46 can be in direct electrical communication with aspects of syringe 70 (e.g., directly connected to plunger 71) rather than running wire 92d from port 46 to tube 61. Voltage wire 92d can be substantially axially aligned along syringe 70 within the disposable cartridge 50. Nozzle tube 92 can be positioned so that a nozzle end of the wire 92d can electrically charge contents of syringe 70 (e.g., by contacting delivery tube 61 as it distally exits luer lock 74) prior to delivery from nozzle assembly 60. For example, distal end of luer lock 74 where delivery tube 61 passes can be electrically connected to a distal end of wire 92d, as shown in FIG. 5B, while an opposite end of the voltage wire 92d (contact end 46) can be in electrical communication with contact end 92a, wire 92c, and port 92e of HV module 86. In some aspects, a wire loop or hook of wire 92d can contact the delivery tube 61. In some aspects, the wire loop or hook of wire 92d can include one or more conductive materials, such as copper, steel, or any other metallic alloy. In some aspects, the inner diameter of the wire loop or hook of wire 92d can be configured to allow for contact with the delivery tube 61. In some aspects, the voltage wire 92d can be attached (e.g., spot welded, crimped, etc.) in place then potted with a high dielectric adhesive. The wire loop or hook of wire 92d can at least partially surround an outer surface of the delivery tube 61 to provide the voltage potential thereto.

In some examples, the NFC tag of disposable cartridge 50 and/or other internal memory of cartridge 50 can include other information about contents (e.g., liquid treatment solution, recommended operational parameters, tracking information, expiry date, etc.) stored therein. This information can be used, for example, by applicator 100 to monitor the type of contents, its volume as well as modify the voltage, flow rate, recommended travel distance (i.e., proximity), etc. for the particular solution. In some example, this information can be stored on an integrated memory, which can include but is not limited to RAM, ROM, EPROM, EEPROM, etc. The information on the integrated memory 120 can be relayed to applicator 100 via on the integrated memory and/or NFC tag, and this information can be used to adjust or otherwise control aspects of cartridge (e.g., components of syringe 70 such as stopper 71a, flow rate, air intake from pump 83, a voltage applied by wire 92d to delivery tube 61, etc.). In some aspects, one or more processors of applicator 100 can be configured to read information of the NFC tag or other internal memory of cartridge 50 related to operational parameters of cartridge 50; and presenting the read information of the NFC in the display screen (e.g., information such as identification of contents of cartridge 50, volume information, etc.). In some aspects, operational information can be written to the NFC by the processor of the electrostatic applicator system.

A distal end of tube 92 can be configured to physically connect with port 69 of nozzle assembly 60. Similarly, air supply tube 76 can run from cartridge air port 59 to nozzle air port 66 of nozzle assembly 60. Tube 76 can provide high velocity air to spray the contents of cartridge 50 (e.g., contents disposed in syringe 70) to the treatment site. The distal end of tube 76 via port 66 can be the outlet of the air supply tube 76 for providing high velocity air flow to nozzle assembly 60.

In some aspects, one or more accumulators (not shown) can be positionable within disposable cartridge 50 or the housing, whereby the one or more accumulators are configured to accept, store, and release energy during operation of the disposable cartridge 50. Examples of one or more accumulators contemplated for use with disposable cartridge 50 may include one or more springs, flywheel energy storage mechanisms, batteries, capacitors, etc.

Turning to FIG. 6, a spring 106 (e.g., a disposably latch spring) can be in communication with a cartridge side of button 57. In operation, spring 106 can provide a spring bias to button 57 that keeps button 57 in the closed position, as shown in the close-up cross section view of section 6 of FIG. 3 which is illustrated in FIG. 6. In some aspects, button 57 as arranged in FIG. 6 with spring 106 can be living hinge configured to maintain button 57 in the depicted closed, retained position. Within the housing side of housing 10, a solenoid 115 can be provided. In FIG. 6, solenoid 115 is depicted in a non-energized position. A solenoid plunger 117 is provided within solenoid 115 and can include a return spring. A solenoid plunger tip 113 can be provided a distal end of plunger 117. As can also be seen, a rigid housing catch 109 is provided so as to retain button 57 in the closed configuration with solenoid 115, including plunger 117 and tip 113. The depicted example of FIG. 6 is but one manner to provide easy, secure attachment and detachment of cartridge 50 with housing 10 of applicator 100 and other attachment approaches are contemplated for use with the disposable cartridge 50 of this disclosure.

FIG. 7A depicts a close-up cross-section view of example aspects of HV wall 93 when cartridge 50 is not connected with housing 10 and FIG. 7B shows example aspects of HV wall 93 when cartridge 50 is connected with housing 10. In FIG. 7A, aspects of HV wall 93 in the air intake and exhaust are closed with respect to housing 10. Specifically, port 147 can include an air path opening through HV wall 93 and ultimately to port 47 of cartridge 50. Though associated tubing of port 147 is not shown, during use tubing would be provided between port 147 and pump 83. Port 147 can include a real valve seal housing in which an intake port 93a of HV wall 93 can be nested. In some aspects, a valve seal conical spring can be positioned to retrain corresponding valve seal 143 in a sealed, closed position, as shown in FIG. 7A. In FIG. 7B, cartridge 50 is shown coupled to HV wall 93. Specifically, port 47 of cartridge 50 is shown inserted into port 93a thereby urging seal 143 to an opened configuration. In turn, air can now pass by seal 143 and into cartridge 50.

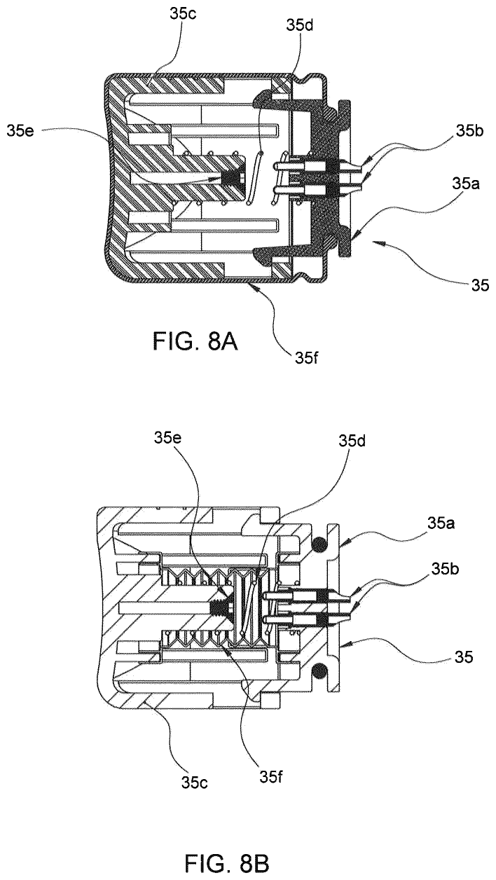

FIG. 8A depicts a side cross-section view of example aspects of actuator 35 previously shown in FIGS. 1A to 3. Specifically, actuator 35 can include a switch base 35a is configured to couple directly to aspects of housing 10. In some aspects, base 35a can be coupled to flexible boot 35f and can be coupled directly switch cap 35c. One or more switch contacts 35b can be positioned in base 35a and in communication with a switch return spring 35d and switch shorting contact 35e. In some aspects, actuator 35 can be configured to resist high-pressure and heavy sprays of water (e.g., an IP6X water dust intrusion rating). In some aspects, the flexible boot 35f is configured to surround components of actuator 35 and allow motion while maintaining sealing.

FIG. 8B depicts a side cross-section view of another example actuator 35′ previously contemplated for use with the examples of FIGS. 1A to 3, where previous flexible boot 35f has been replaced by flexible internal bellow boot 35′ positioned internal to switch cap 35c and directly coupled to base 35a. In some aspects, flexible internal bellow boot 35′ is configured to surround return spring 35d and contact 35e so as to resist high-pressure and heavy sprays of water (e.g., an IP6X water dust intrusion rating).

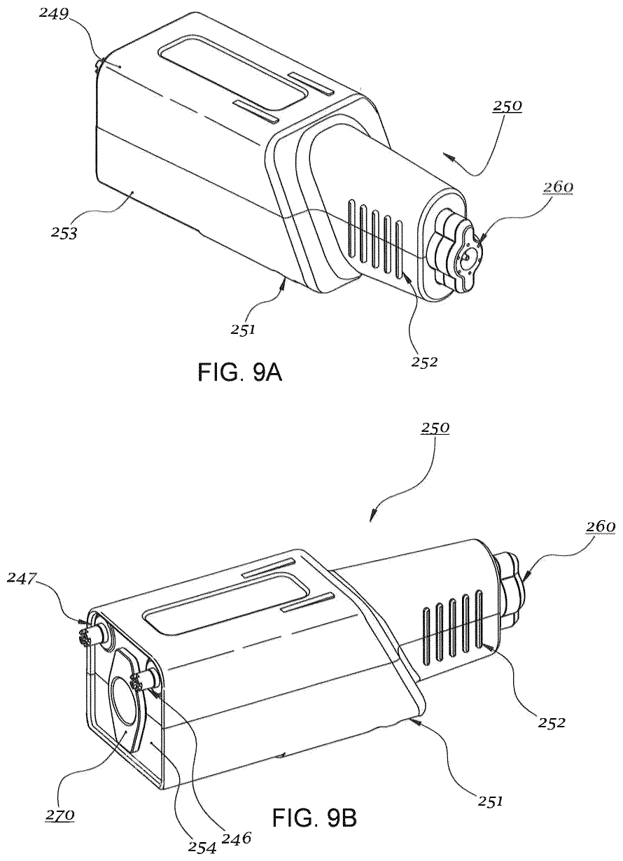

Referring to FIG. 9A, a forward perspective view of a cartridge 250 configured for electrospinning is provided while FIG. 9B depicts a rear perspective view of cartridge 250. Aspects of cartridge 250 include like numerals as in cartridge 50 indicate like structural elements and features. In some aspects, nozzle 260 of cartridge 250 is configured to receive source liquid from syringe 270 and feed high-compressed air via one or more injection holes 267 so that the source liquid of syringe 270 for fiber can be injected together with air and a voltage applied thereto to produce and emit electrospun fibers having fine diameters. In some aspects, holes 267 can be radially arranged around tube 261 and formed in FIG. 9B specifically shows the supply end and contact end of cartridge 250. HV contact 246 is positioned on a face of cartridge HV wall 254, which is configured to mount against HV wall 93 of housing 10. HV wall 254 can also include an air intake port 247 which can be configured to couple to a corresponding air intake port 147 of HV wall 93 (see, e.g., FIGS. 7A and 7B).

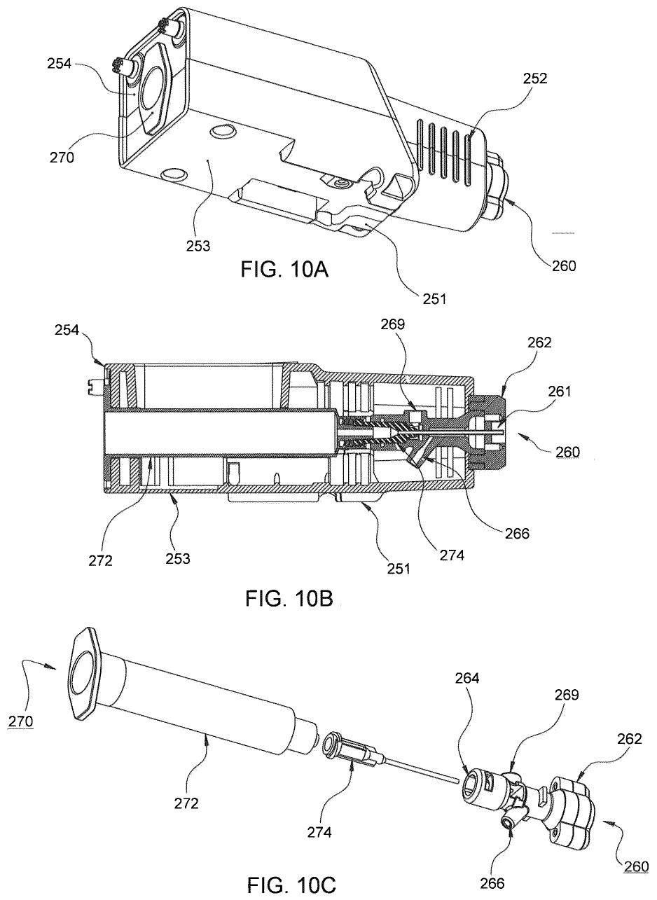

Similarly, FIG. 10A depicts a lower rear perspective view of cartridge 250. FIG. 10B depicts a side view of cartridge with a portion of its outer housing 249 removed while FIG. 10C depicts an exploded perspective view of internal components of cartridge 250 with outer housing 249 removed. Similar to housing 49, housing 249 can be formed and/or assembled in a number of ways, including but not limited to, machining, molding, injection molding, three-dimensional printing, or any other suitable manufacturing process. Suitable materials for housing 249 may include one or more of glass filled nylon, glass filled polypropylene, glass filled polyethylene, polypropylene, polyethylene, or a plastic material.

As shown in FIGS. 9A, 10B, and 10C, nozzle assembly 260 can include nozzle housing 260a. Housing 260a can include nozzle outlet channel 262 on a distal end that can contain the outlet end of delivery tube 261. The nozzle housing 260a can include air inlet port 266 in fluid communication with holes 267 positioned about channel 262. As shown in FIG. 9A, housing 260a can include six (6) holes radially arranged around a common axis of channel 262, though fewer or greater number of holes can be used as needed or required. In some aspects, holes 267 can be replaced by an outer concentric outlet channel that completely surrounds channel 262 and is similarly in fluid communication with port 266. During use, air pumped from port 266 and out through holes 267 can create an air curtain, around the electrospun fiber being emitted from tube 261. In the depicted example of FIG. 9A, holes 267 are shaped substantially straight or otherwise angled in parallel and/or axially aligned with tube 261 so that the shape of the corresponding air curtain is similarly parallel and/or axially aligned. In other examples, holes 267 are shaped angled upwards relative to a longitudinal axis of tube 261 so as to create a funnel shaped, tapered outward air curtain. In other examples, holes 267 are shaped angled downwards relative to a longitudinal axis of tube 261 so as to create a converging, tapered inward shaped air curtain.

In some examples, housing 249 can comprise two or more sections of moldable plastic, e.g., a first half and a second half (which, when assembled together will be referred to herein as housing 249). The two or more sections can be assembled together with fasteners (e.g., screws, rivets, a weld [e.g., a sonic weld], one or more straps or snaps, an adhesive or adhesive tape, etc.) such that the internal components are disposed between the portions and/or respective halves of housing 249. In some aspects, housing 249 can include a fiber outlet associated with nozzle assembly 260 that enables the atomized, charged treatment solution to be expelled from cartridge 250. Port 247 is configured to receive air supply tube 81 to place tubes 81, 76 and pump 83 in fluid communication with each other.

Just as with cartridge 50, cartridge 250 can securely engage with chamber 27. Though not shown, cartridge 250 can contain, within its housing 249, an air supply tube, voltage tube, and syringe 270. Similar to syringe 270, syringe 270 can be an assembly that includes a glass or plastic syringe for storing the liquid solution that is to be applied using the disposable cartridge 250. Syringe 270 can be assembled within the disposable cartridge 250 pre-filled with the desired liquid solution. At a proximal end, syringe 270 can include a plunger and a barrel portion 272 extended distally therefrom. Contents of syringe 270 can be advanced through luer lock 274 positioned at distal end of portion 272. An inner distal delivery tube 261 can be distally extended from luer lock 274 through nozzle assembly 260. At a distal end of luer lock 274, a distal end of a voltage wire (not shown, though similar to wire 92d of cartridge 50) can run through the nozzle tube of cartridge 250 and be connected to a proximal portion of inner distal delivery tube 261, which can be constructed from one or more conductive materials. In operation, syringe 270 can be configured to deliver the electrospun fiber from the stored liquid solution at a predetermined rate from barrel portion 272 through delivery tube 261 and ultimately emitted via nozzle assembly 260 onto a treatment site (e.g., a wound site of a patient).

In some examples, the previously mentioned NFC tag of disposable cartridge 250 and/or other internal memory of cartridge 250 can include other information about contents (e.g., liquid treatment solution) stored therein. This information can be used, for example, by applicator 100 to monitor the type of contents, its volume as well as modify the voltage, flow rate, recommended travel distance (i.e., proximity), etc. for the particular solution. In some example, this information can be stored on an integrated memory, which can include but is not limited to RAM, ROM, EPROM, EEPROM, etc. The information on the integrated memory 120 can be relayed to applicator 100 via on the integrated memory and/or NFC tag, and this information can be used to adjust or otherwise control aspects of cartridge (e.g., components of syringe 270).

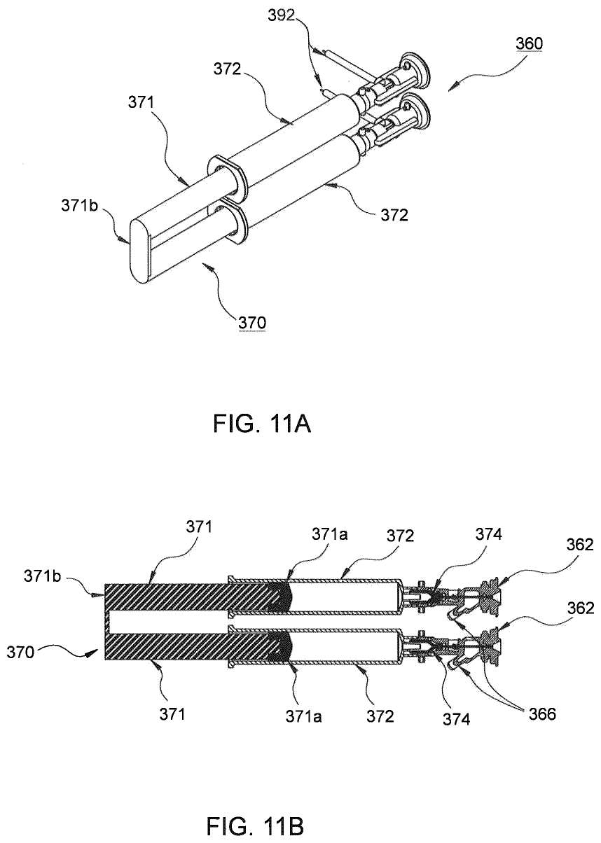

FIG. 11A depicts a perspective view of internal components of another example electrostatic cartridge 350 contemplated for use with the example electrostatic applicator of FIGS. 1A to 3, the components including a pair of syringe and electrostatic nozzles. FIG. 11B depicts a side cross-section view of the example internal components shown in FIG. 11A. The outer housing of cartridge 350 is not shown in FIGS. 11A and 11B strictly for illustrative purposes to observe these internal components. As opposed to previously described cartridges 50 and 250, cartridge 350 can contain, within its housing, a multi-plunger syringe 370 whereby each sub-syringe of multi-plunger syringe 370 can include its own air supply tube, voltage tube 392, barrel portion 372, stopper 371a, and syringe actuating shaft 371. Actuating shafts 371 of multi-plunger syringe 370 can be connected proximally via a central drive surface 371b so that advancing surface 371b causes each connected actuating shaft 371 to simultaneously advance respective stopper 371a to distally urge contents stored in respective portion 372 through respective luer locks 374 and ultimately into respective nozzle assemblies 360. In some aspects, the voltage tubes 392 and respective voltage wires (not shown) of the depicted examples in FIGS. 11A and 11B can include separate and independent high voltage inputs. In some aspects, the example of FIGS. 11A and 11B is configured to increase the production of deliverable treatment solution therefrom (e.g., electrosprayed contents, electrospun fiber, etc.) as well as cover greater areas faster via multiple nozzle assemblies with the high voltage separated and independent all the way through the fluid path, as shown. In some aspects, syringe 370 can be configured so that sub-assemblies dispense in different ratios other than 1:1. While only two sub-assemblies are shown in FIGS. 11A and 11B, it is contemplated that more than two sub-assemblies can be used as needed or required.

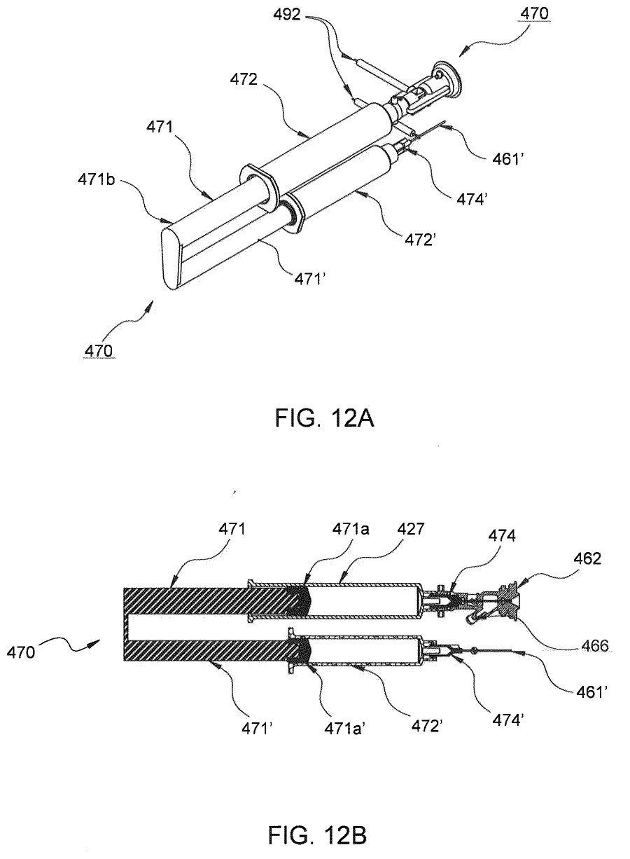

FIG. 12A depicts a perspective view of internal components of another example electrostatic cartridge 450 contemplated for use with the example electrostatic applicator of FIGS. 1A to 3, the components including a pair of syringe and electrostatic nozzles. FIG. 12B depicts a side cross-section view of the example internal components shown in FIG. 12A. The outer housing of cartridge 450 is not shown in FIGS. 12A and 12B strictly for illustrative purposes to observe these internal components. In some aspects, the example of FIGS. 12A and 12B is configured to increase the production of deliverable treatment solution therefrom (e.g., electrosprayed contents, electrospun fiber, etc.) as well as cover greater areas faster via multiple nozzle assemblies with the high voltage separated and independent all the way through the fluid path, as shown. In some aspects, a first subassembly of syringe 470 can include rod 471, stopper 471a, barrel portion 472, luer lock 474, and nozzle assembly 460, collectively which are configured for electrospraying. In some aspects, a second subassembly of syringe 470 can include rod 471′, stopper 471a′, barrel portion 472′, luer lock 474′, and electrospinning tube 461′, collectively which are configured for electrospinning. In some aspects, In some aspects, syringe 470 can be configured so that sub-assemblies dispense in different ratios other than 1:1. In some aspects, use of each of the first and second subassemblies of syringe 470 is advantageous to utilize both electrospraying and electrospinning with a single cartridge. In some aspects, barrel portions 472 and 472′ can be configured to contain a different volume and/or type of fluid solution.

As opposed to previously described cartridges 50 and 250, cartridge 450 can contain, within its housing, a multi-plunger syringe 470 whereby each sub-syringe of multi-plunger syringe 470 can include its own air supply tube, voltage tube 492, barrel portion 472, 472′, stopper 471a, 471a′, and syringe rod 471, 471′. Shafts 471, 471′ of multi-plunger syringe 470 can be connected proximally via a central drive surface 471b so that advancing surface 471b causes each connected rod 471, 471′ to simultaneously advance respective stopper 471a, 471a′ to distally urge contents stored in respective portion 472, 472′ through respective luer locks 474, 474′ and ultimately into respective nozzle assemblies. In some aspects, the voltage tubes 492 and respective voltage wires (not shown) of the depicted examples in FIGS. 12A and 12B can include separate and independent high voltage inputs. While only two sub-assemblies are shown in FIGS. 12A and 12B, it is contemplated that more than two sub-assemblies can be used as needed or required.

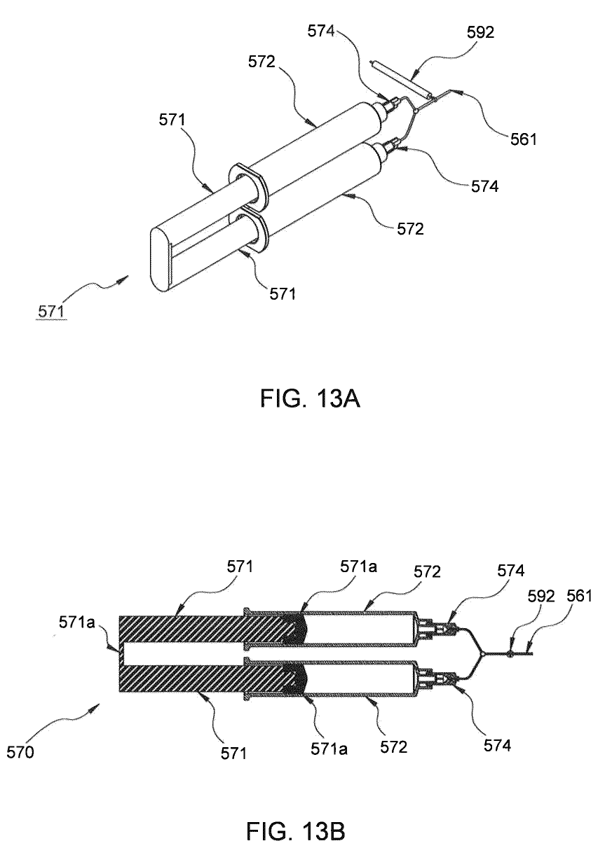

FIG. 13A depicts a perspective view of internal components of another example electrostatic cartridge 550 contemplated for use with the example electrostatic applicator of FIGS. 1A to 3, the components including a pair of syringe and electrostatic nozzles. FIG. 13B depicts a side cross-section view of the example internal components shown in FIG. 13A. The outer housing of cartridge 550 is not shown in FIGS. 13A and 13B strictly for illustrative purposes to observe these internal components. As opposed to previously described cartridges 50 and 250, cartridge 550 can contain, within its housing, a multi-plunger syringe 570 whereby each sub-syringe of multi-plunger syringe 570 can include its own air supply tube, voltage tube 592, barrel portion 572, stopper 571a, and syringe rod 571. Actuating shafts 571 of multi-plunger syringe 570 can be connected proximally via a central drive surface 571b so that advancing surface 571b causes each connected rod 571 to simultaneously advance respective stopper 571a to distally urge contents stored in respective portion 572 through respective luer locks 574 and ultimately into respective nozzle assemblies 560.

In the example of FIGS. 13A and 13B, each of barrel portions 572 and corresponding subassembly structure can be interconnected by a Y-shaped delivery needle 561. Tube 561 as shown can include proximal ports in fluid communication with each luer lock 574 and barrel portions 572 and join in a single distal end through solution can be emitted or otherwise exit. Voltage tube 592 and corresponding voltage wire (not shown) can couple to delivery tube 561 proximal of the distalmost end of tube 561 so that all solution emitted therefrom is charged by the voltage wire. While a Y-shaped member is shown, any number of syringe subassemblies can be used as required with cartridge 550. In some aspects, the voltage tubes 592 and respective voltage wires (not shown) of the depicted examples in FIGS. 13A and 13B can include separate and independent high voltage inputs. In some aspects, the example of FIGS. 13A and 13B is configured to increase the production of deliverable treatment solution therefrom (e.g., electrosprayed contents, electrospun fiber, etc.) as well as cover greater areas faster via multiple nozzle assemblies with the high voltage separated and independent all the way through the fluid path, as shown. In some aspects, syringe 570 can be configured so that sub-assemblies dispense in different ratios other than 1:1. While only two sub-assemblies are shown in FIGS. 13A and 13B, it is contemplated that more than two sub-assemblies can be used as needed or required.

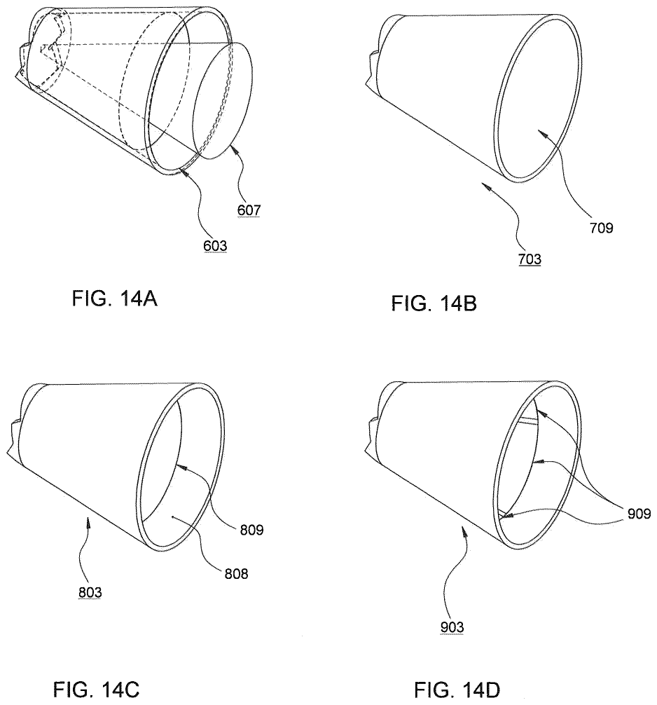

FIG. 14A depicts a side perspective view of an example cone 607, 603 configured for use with any of the herein disclosed nozzle assemblies of the described disposable cartridges. In some aspects, cone 603 can be a physical cone of any material that attaches to either a distal end of the nozzle assembly or directly to the housing 10. In some aspects, cone 607 can be attached directly to a distal end of a delivery tube associate with the respective nozzle assembly. In some aspects, cone 603 can be configured to protect the emission of electrospraying or electrospinning materials from the respective system (e.g., applicator 100 and any disposable cartridge connected thereto) to the treatment site (e.g., a wound site of the patient) by preventing external air flow and/or external forces from interfering. In some aspects, cone 603 can be configured to control airflow in the respective nozzle assembly by reflecting spray off of its cone walls. In some aspects, once attached to housing 10 and/or a respective nozzle assembly, cone 603 can be configured so that one or more lights (e.g., LED lights) can ring the proximal end of the cone from the electrostatic applicator and generate a light pattern (e.g., a light ring) via the light pipe effect on the target site to aide targeting. In some aspects, if a translucent material is used with cone 603, the cone can disperse the light, including different colors, from applicator 100 to notify the user of proper distance by warning if the applicator is too close or too far.

FIG. 14B depicts a side perspective view of another example cone 703 configured for use with any of the herein disclosed nozzle assemblies of the described disposable cartridges. In some aspects, cone 703 can be a physical cone of any material that attaches to either a distal end of the nozzle assembly or directly to the housing 10. Similarly, cone 703 can illuminate when solution is being emitted therefrom. For example, inner surface 709 of cone 703 can be configured to illuminate as well as be plated or finished with a non-stick surface.

FIG. 14C depicts a side perspective view of another example cone 803 configured for use with any of the herein disclosed nozzle assemblies of the described disposable cartridges. In some aspects, cone 803 can be a physical cone of any material that attaches to either a distal end of the nozzle assembly or directly to the housing 10. In some aspects, inner surface 809 of cone 803 can be plated or finished with a conductive surface that can have the same polarity as the fluid being dispensed that would help with the dispensing direction. A distal edge 808 of cone 803 can be left without that same conductive surface plating and/or finishing.

FIG. 14D depicts a side perspective view of another example cone 903 configured for use with any of the herein disclosed nozzle assemblies of the described disposable cartridges. In some aspects, cone 903 can be a physical cone of any material that attaches to either a distal end of the nozzle assembly or directly to the housing 10. In some aspects, inner surface 909 of cone 903 can be plated or finished with multiple conductive surfaces (e.g., radially separated surfaces that can be charged to repel and/or attract the solution and change dispensing directions).

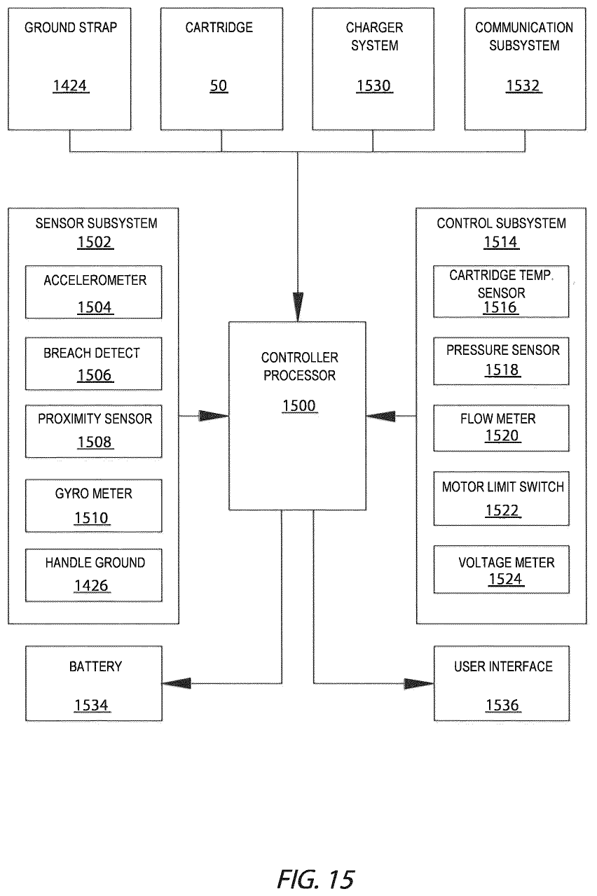

FIGS. 15-17 are component diagrams of various components and systems that can be included in an electrostatic applicator system (for example within applicator 100 and/or any of the disposable cartridges 50, 250, 350, 450, 550), according to the present disclosure. A controller 1500, which can include a the CPU, can communicate with the various components and systems to operate the electrostatic applicator. For example, the controller 1500 can communicate with the disposable cartridge in order to receive information about the particular fluid contained within the cartridge. This information communicated to the controller 1500 (e.g., via integrated memory) can include information about preferred fluid flow rates (e.g., by adjusting the motor speed), air supply rates, voltages (e.g., by adjusting the voltage potential between the voltage supply (e.g., HV module 86) and delivery tube of the respective nozzle assembly of the respective cartridge) and the like such that preferred nanoparticles can be formed from the sprayed therapeutic solution. This information enables a single reusable applicator 100 to be used with numerous different disposable cartridges 50, 250, 350, 450, 550with different solutions. Further, not only are the fluids potentially different in each disposable cartridge 50, 250, 350, 450, 550. For example, different disposable cartridges 50, 250, 350, 450, 550 can have different air supply tubes, voltage wires, and/or nozzle assemblies depending on the particular application for the disposable cartridge 50, 250, 350, 450, 550. To this end, this component information can also be relayed to controller 1500 so that the reusable applicator 100 can be adjusted accordingly.

The controller 1500 can communicate with a sensor subsystem 1502 that includes various sensors that can be used to operate the reusable applicator 100. The sensor subsystem 1502 can include an accelerometer 1504 that can be used to wake the CPU when the user moves the reusable applicator 100. For example, in addition to or as an alternative to any button or capacitive input from an associated user interface described above, the reusable applicator 100 can automatically turn on (e.g., the CPU can receive power) when the accelerometer detects movement of the reusable electrostatic applicator. The sensor subsystem 1502 can include a breach detect 1506 located proximate cartridge chamber 27 to determine if the cartridge is sufficiently attached to chamber 27. The breach detect 1506 can act as a safety measure to ensure the disposable cartridge 50, 250, 350, 450, 550 is fully seated and within chamber 27 before applicator 100can be activated. The breach detect 1506 can be a pressure sensor, switch, and/or the like.

The sensor subsystem 1502 can include a proximity sensor 1508 to detect how close the reusable applicator 100 is to the treatment or target site. The proximity sensor 1508 can be positioned at the distal end of the reusable applicator 100, for example proximate or adjacent the spray outlet of the respective cartridge 50, 250, 350, 450, 550. Alternatively, the proximity sensor 1508 can be positioned on the disposable cartridge 50, 250, 350, 450, 550 proximate or adjacent the housing spray outlet of the respective cartridge 50, 250, 350, 450, 550. The proximity sensor 1508 can include but is not limited to inductive proximity sensors, capacitive proximity sensors, photoelectric proximity sensors, and the like. The proximity sensor 1508 can be a safety feature (e.g., configured to detect proximity between the electrostatic applicator and the target) used to indicate to the operator whether the electrostatic applicator is within a preferred distance to the target object. For example, the travel distance of the charged droplets can affect the morphology of the droplets as they contact the target site. To this end, the proximity sensor 1508 can transmit signals to the controller 1500 to indicate the distance to the target object, and the controller 1500 can output a signal to a user interface 1536 (e.g., a display screen, an external user device, etc.) to alert the user if the device is too far away from or too close to the target site. This information can be based upon the information stored on integrated memory of the disposable cartridge 50, 250, 350, 450, 550, as described above.

The sensor subsystem 1502 can include a gyrometer 1510 (or gyroscope) that can be used to help to measure or maintain a certain positioning of the reusable applicator 100. For example, the gyrometer 1510 can output a signal to the user interface 1536 (e.g., the display screen, an external user device, etc.) to indicate to the user that the device should be moved to an upright configuration or any other configuration. The sensor subsystem 1502 can also include the handle ground 1426 described above. In addition to grounding the operator, the handle ground 1426 can be used to detect if a user is holding the device and whether the device should be activated.

The controller 1500 can communicate with a control subsystem 1514 that includes various sensors, switches, and the like that can be used to ensure the reusable applicator 100 is operating as expected. The control subsystem 1514 can include a cartridge temperature sensor 1516. Although it is contemplated that the disposable cartridge 50, 250, 350, 450, 550 can be stored and used at room temperature, the cartridge can also be stored in other conditions, for example in a frozen state to preserve the therapeutic solution stored therein. The cartridge temperature sensor 1516 can detect the temperature of the therapeutic solution and alert the user if the solution is too cold, or too hot, to be administered to a patient's skin.

The control subsystem 1514 can include a pressure sensor 1518 positioned to read the air pressure of air flow through the air supply into the respective cartridge (e.g., the air supply associated with pump 83). This pressure information can be used by the controller 1500 to determine if the air flow through the device is providing the preferred air velocity for the particular fluid being sprayed. The control subsystem 1514 can include a flow meter 1520 positioned to read the fluid flow rate of the fluid travelling through or out of the nozzle of the respective cartridge 50, 250, 350, 450, 550. This flow rate information can be used by the controller 1500 to determine if the fluid flow through the device is providing the fluid volume for the particular solution being sprayed.

The control subsystem 1514 can include a motor limit switch 1522. The motor limit switch 1522 can be used to define the rate at which the piston of the applicator is actuated to further define and/or modulate the flow rate of the treatment solution. The control subsystem 1514 can include a voltage meter 524. The voltage meter 524 can be used to determine what voltage is being applied at the nozzle tube 106. If the voltage is off for any reason for the particular fluid, an alert can be transmitted to the user interface 1536 (e.g., the display screen of user interface 87, an external user device, etc.).

In some aspects, the control subsystem 1514 can include or be in communication with a cartridge detect switch configured to detect presence of a cartridge when assembled with the electrostatic applicator. If presence is detected, one or more operations related to a respective cartridge can be performed or otherwise initiated. In some aspects, the control subsystem 1514 can include or be in communication with an NFC chip of disposable cartridge, whereby the NFC chip can include an integrated memory with information related to characteristics of contents (e.g., treatment solution) stored within a syringe of and/or within the cartridge, including operating parameters of the contents of the cartridge such as whether the cartridge is sealed or unsealed, flow rate, a voltage potential, and/or nozzle setting associated with nozzle, so as to adjust a motor speed or a applied voltage to the contents for deposition to the treatment site from the cartridge and/or electrostatic applicator.

The controller 1500 can also receive signals indicating whether the ground strap 424 has been applied to an external ground (e.g., the wound site of a patient and/or target). If not, the controller 1500 can transmit an alert to the user interface 1536 and/or prohibit activation of the reusable applicator 100 (e.g., preventing, for example via a switch, an electrical connection between the voltage supply of HV module 86 and the respective nozzle of the respective cartridge 50, 250, 350, 450, 550). The controller 1500 can also communicate with a charger system 1530. As described above, the voltage supply 420 can include one or more batteries that can be rechargeable. The charger system 1530 can include AC/DC converter(s) and an inductive integrated circuit(s) so as to manage charging of the rechargeable voltage supply of HV module 86.

The controller 1500 can also communicate with a communication subsystem 1532. The communication subsystem 1532 can be integrated within the CPU and/or HV module 86 and include one or more transceivers that can communicate with external devices. The one or more transceivers can be compatible with short range wireless communication connections, for example but not limitation radio-frequency identification (RFID), near field communication (NFC), Bluetooth™, low-energy Bluetooth™ (BLE), WiFi™, ZigBee™, and/or similar connections. The communication subsystem 1532 can enable the reusable applicator 100 to communicate with an external device, such as a user device. The user device can include a mobile cellular device, personal digital assistant (PDA), tablet, laptop or desktop computer, smart wearable device, and/or the like. In this example, the user device can operate similar to the display screen to provide information to the device operator. For example, a display screen of a user device can be used in addition to or as an alternative to a display screen. The wireless communication between the reusable applicator 100 and a user device can provide an option to use the user device as an actuator to activate the spraying sequence of the electrostatic applicator. For example, an activation input to the controller 1500 (or the CPU) can include providing an input into a display screen of the external user device.

Additionally, information related to the fluid within the disposable cartridge 50, 250, 350, 450, 550 can be input into the user device to inform the controller 1500 which spray parameters are to be used for the particular cartridge. In some examples, a barcode, Quick Response (QR) code, and the like can be placed on the disposable cartridge 50, 250, 350, 450, 550. A user can scan the code with a camera or other scanner on the user device, and the information can be relayed to the controller 1500. The controller 1500 can then determine the necessary spraying parameters for the particular cartridge. The user can then input an activation input (e.g., press an icon on the screen of the user device) to begin spraying the treatment solution.

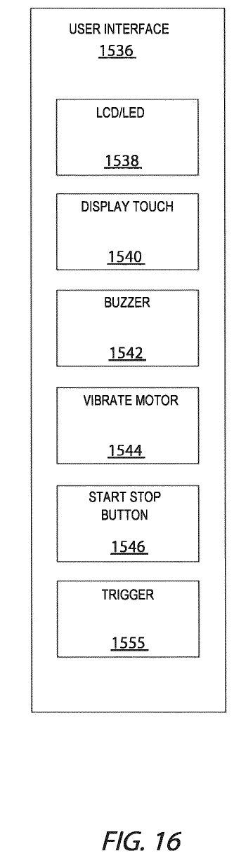

Referring again to FIG. 15, the controller 1500 can communicate with a user interface 1536 that can provide information to and receive information from an operator of the reusable applicator 100. FIG. 16 provides a detailed component diagram of an example user interface 1536. The user interface 1536 can include the display screen of user interface 87 described above. For example, the user interface 1536 can include an LCD/LED screen 1538 for providing information about the status of the device to the user. In some examples, the user interface 1536 can include a display touch panel 1540. In the examples where the user interface 1536 (e.g., prior user interface 87) is a display screen on the reusable applicator 100, the display screen can include touchscreen capabilities. This can enable the display screen to receive an activation input to initiate the spray sequence for the reusable applicator 100. For example, the display screen can provide an icon that can act as a virtual “actuator” to activate the spray sequence. Further, information about the fluid contained in a particular disposable cartridge 50, 250, 350, 450, 550 can be inputted into this display touch panel 1540 so as to inform the controller 1500 (or the CPU) what parameters should be used for the particular treatment solution.

The user interface 1536 can include a buzzer 1542. The buzzer 1542 can include one or more speakers that can indicate to the operator whether an issue should be addressed with respect to the device. For example, if the disposable cartridge 50, 250, 350, 450, 550 has not been fully seated, if a ground of applicator 100 (e.g., a ground strap 1424 or handle ground 1426) does not detect a proper ground, if the voltage supply of HV module 86 is low on power or should be charged, etc., the buzzer 1542 can provide audible feedback on the particular status. Further, the display screen of user interface 87 can also provide visual feedback to additionally alert the user to the particular issue. In addition to or alternatively, the user interface 1536 can include a vibrate motor 1544 that can indicate, via tactile feedback to the user, when an issue occurs. The user interface 1536 can include a start/stop button 1546. Alternatively, the start/stop button 1546 can be integrated into a display screen, for example display screen of user interface 87 or a display screen on an external user device, so that the activation of the device can be performed within a screen that has touchscreen capabilities. The user interface 1536 can include an actuator 1555 (e.g., a trigger). As described above, the actuator 1555 can be an actuator, a virtual actuator within a user interface of an external user device, or a virtual actuator as an icon in the display screen of the user interface 87.

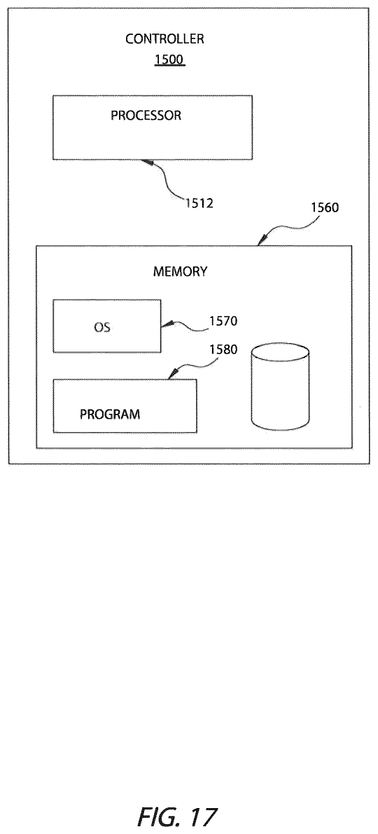

FIG. 17 is a component diagram of an example controller 1500, according to the present disclosure. As described above, the controller 1500 can include one or more processors, e.g., the CPU. The CPU can include one or more of a microprocessor, microcontroller, digital signal processor, co-processor and/or the like or combinations thereof capable of executing stored instructions and operating upon data. The CPU can constitute a single core or multiple core processor that executes parallel processes simultaneously. For example, the CPU can be a single core processor that is configured with virtual processing technologies. The CPU can use logical processors to simultaneously execute and control multiple processes.

The controller 1500 can include a memory 1560. The memory 1560 can be in communication with the one or more processors (e.g., the CPU). The memory 1560 can include instructions, for example a program 1580 or other application, that causes the CPU and/or controller 1500 to complete any of the processes described herein. For example, the memory 1560 can include instructions that cause the controller 1500 and/or the CPU to receive an activation signal (e.g., from a manual trigger, from an external user device, from a display screen of user interface 87, etc.), output a control signal (e.g., a control signal to the motor and/or piston associated with actuating urging of solution from a respective cartridge through a respective nozzle) to actuate to deliver fluid from the syringe 70, 270, 370, 470, 570, output a control signal to a switch to provide voltage from the voltage supply of HV module 86 to the respective voltage wire (e.g., wire 92d).

The memory 1560 can include, in some implementations, one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs, executable instructions and data. The memory 1560 can also include a program, e.g., program 1580, that includes the instructions to complete the processes described herein. For example, the program 1580 can include instructions to receive the activation signal (e.g., from actuator of applicator 100, or a virtual trigger, for example from an external user device and/or from the display screen of user interface 87), output a first control signal to the motor to actuate a piston associated with actuating a respective cartridge, and/or output a second control signal to a switch to provide voltage from the voltage supply of HV module 86 to the voltage wire. Further, the controller 1500 can include data storage 1590 that can store data associated with, for example, the parameters in which to adjust the air flow, fluid flow, and/or voltage for the particular treatment solution.

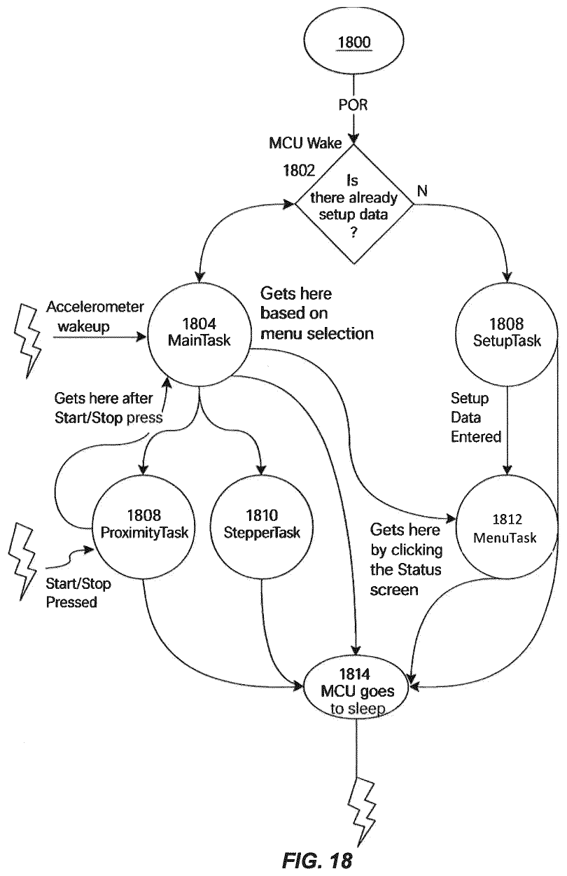

FIGS. 18-21 are flow diagrams of example processes for operating a reusable applicator 100/disposable cartridge 50, 250, 350, 450, 550 system, according to the present disclosure. Specifically, FIG. 18 is a flow chart of an exemplary process 1800 to manage operations of the herein described applicator 100. In process 1800, the microcontroller unit can wake up to determine if setup data is present (step 1802). If yes, then applicator 100 system can be woken to execute the main task process (step 1804). The main task process of step 1804 can include proximity task step 1808, stepper motor task 1810, menu task 1812, and MCU sleep mode 1814. In some aspects, main task of step 1804 can be initiated upon detecting of input from an accelerometer in communication therewith (e.g., in response to detecting a predetermined change in acceleration above a threshold indicative of user movement of applicator 100). In some aspects, proximity task 1808 can be initiated when a corresponding proximity sensor (e.g., target sensor 45) detects that a target site is within a threshold sprayer range of applicator 100. In some aspects, proximity task step 1808 can be initiated by starting and/or stopping actuation of an associated actuator (e.g., a button or capacitive input of the display screen of user interface 87).

If in step 1802 the system determines that there is no setup data present, then step 1806 of the setup task of applicator 100 is executed. In some aspects, step 1806 can include setup data being entered (e.g., via a display screen of user interface 87). Upon setup data being entered, menu task 1812 can be executed. If actuation or other input has not been received within a predetermined time period, then in step 1814 the system will enter the MCU sleep mode.

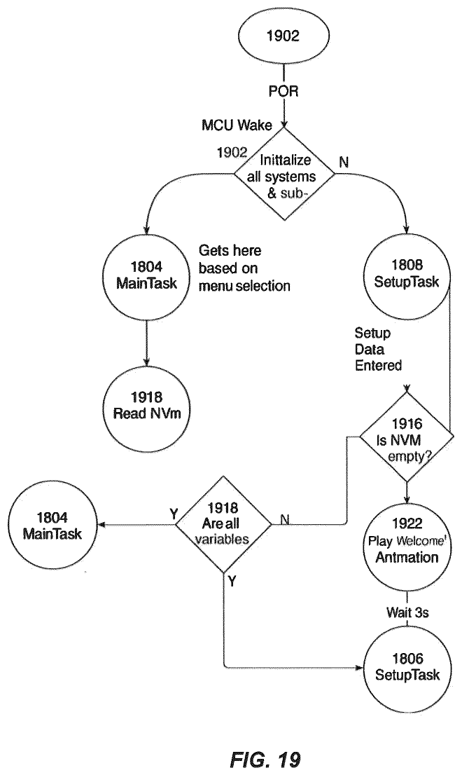

FIG. 19 is a flow chart of an exemplary process 1900 utilizing certain aspects from process 1800. In process 1900, the microcontroller unit can wake up to initialize all systems and subsystems of applicator 100 (step 1902). Such initializing can include initiating setup tasks 1904, menu tasks 1906, main tasks 1908, proximity tasks 1910, stepper motor task 1912, as well as other initializing tasks. After completing step 1902, then the step of reading non-volatile memory 1914 is executed and if the non-volatile memory is not empty (step 1916), then the system will determine whether all variables are loaded (step 1918). If all variables are loaded, then the previously discussed main task 1804 of process 1800 can be performed. If the non-volatile memory is empty in step 1916, then the system can play a welcome animation on the display screen of interface 87 (step 1922). Similarly, if it is determined in step 1918 that all variables are not loaded, then step 1922 can similarly be executed by the system. Upon completion of step 1922 in either context and/or after waiting a predetermined period of time (e.g., approximately 3 seconds), previous setup task step 1806 from process 1800 can be executed.

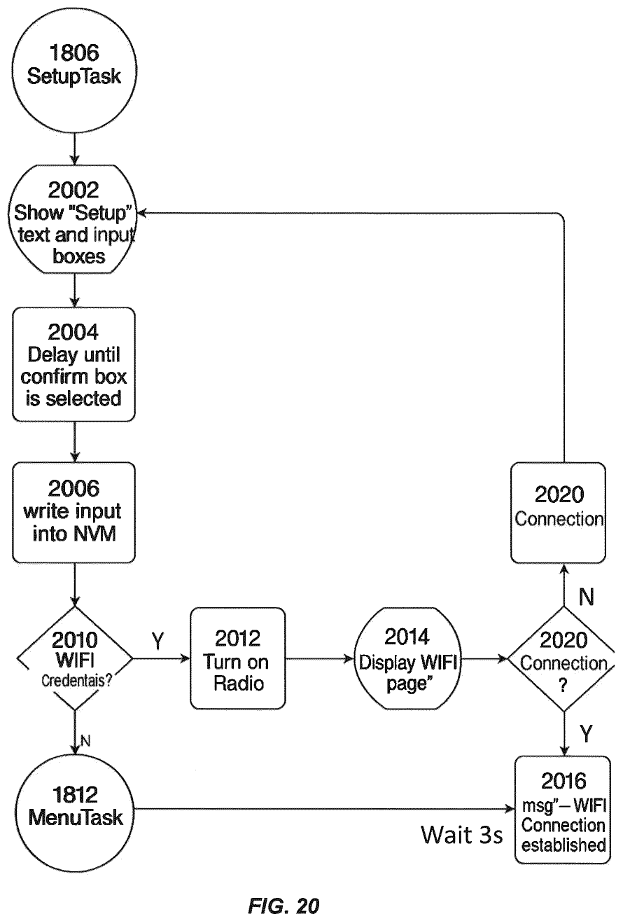

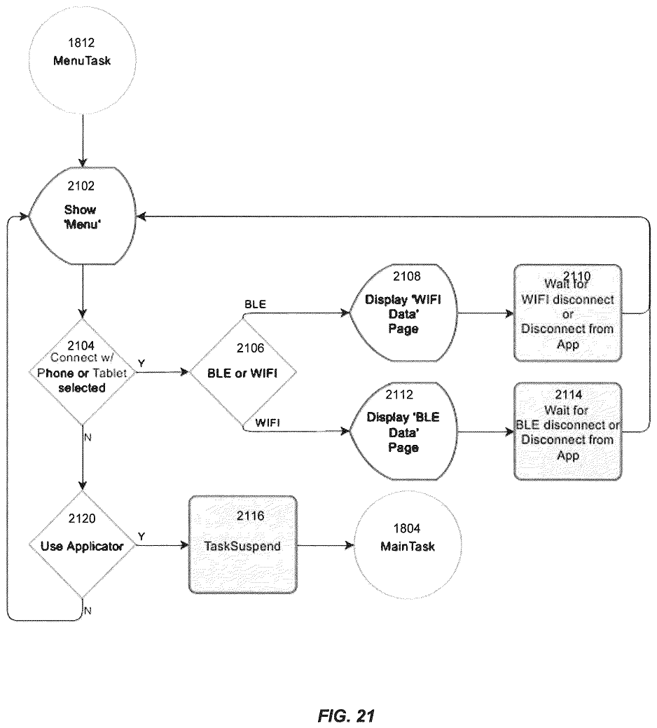

FIG. 20 is a flow chart of an exemplary process setup task 1806 of previous process 1800 as to initiating wireless connectivity between an example applicator 100 and a wireless network.

FIG. 21 is a flow chart of an exemplary process setup task 1812 of previous process 1800 as to initiating wireless connectivity between an example applicator 100 and an external device so that the external device (e.g., a mobile computing device such as a smart phone, a tablet, etc.), can control and/or monitor one or more operations of aspects of applicator 100, including but not limited to operations of a cartridge coupled thereto.

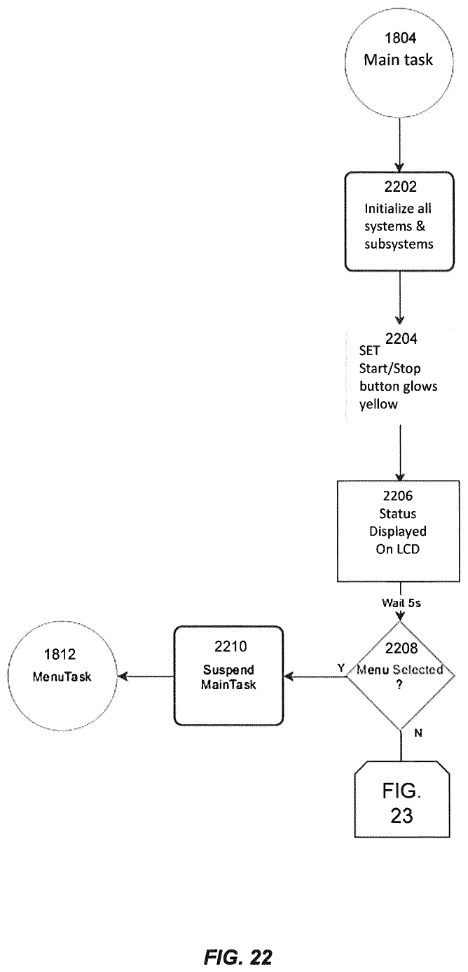

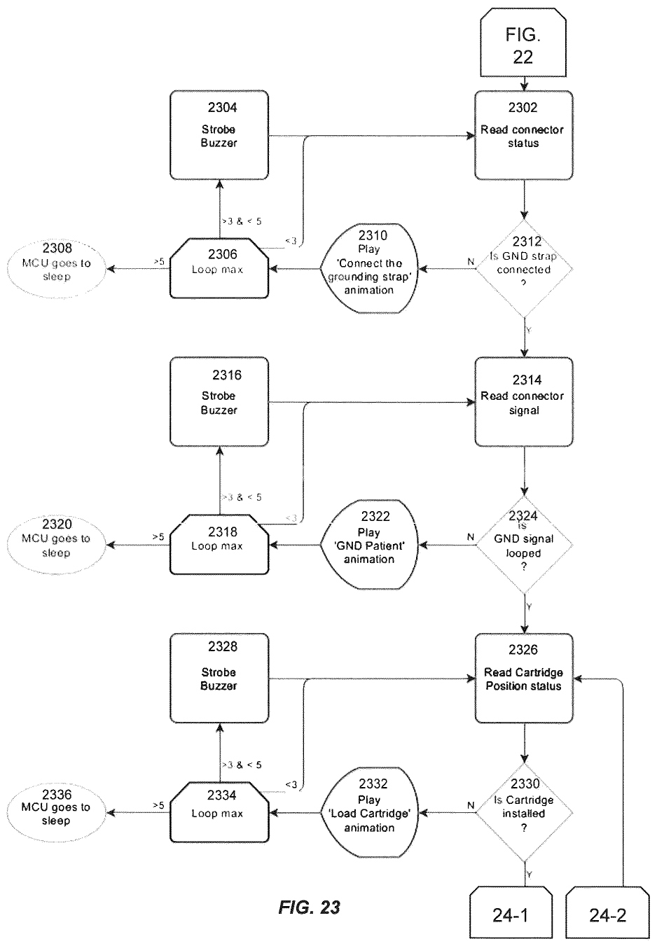

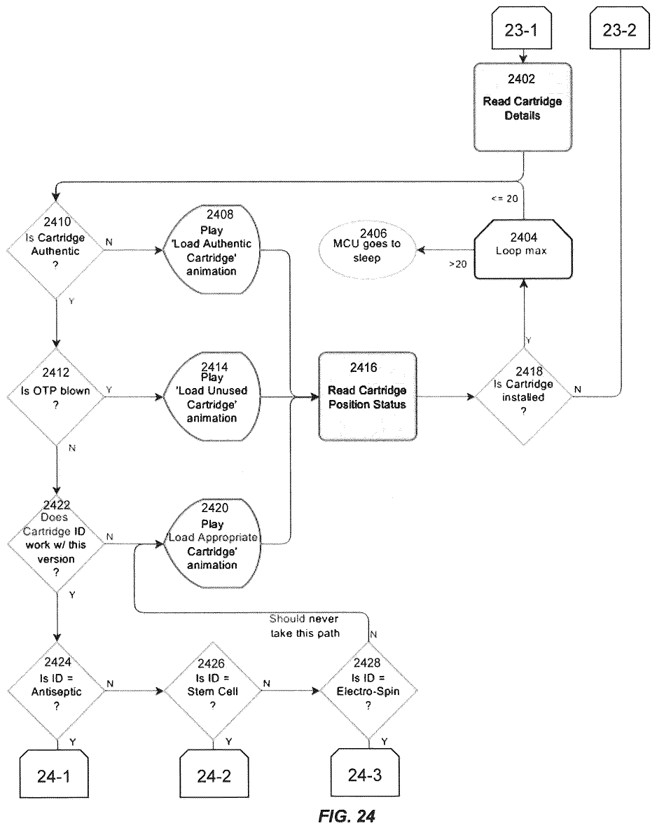

FIG. 22 is a flow chart of an exemplary process main task 1804 of previous process 1800 as to initializing all systems and subsystems of an example applicator 100. FIG. 23 is a flow chart that continues from FIG. 22 and specifically described example substeps related to determining and managing operations related to a ground strap and any related ground signal, in those examples where a ground strep is present. FIGS. 23 and 24 also describes exemplary steps related to operations of loading and managing assembly of any heretofore described cartridge with the example applicator 100 of this disclosure. For example and without limitation, in step 2330 of FIG. 23, the cartridge position can be analyzed to determine whether it has been properly installed. If not, then an animation can be played (step 2332) until ultimately the MCU can cause a sleep mode to execute (step 2336). If the cartridge is determined to be properly installed (step 2330), then in step 2402 cartridge details can be read to determine certain information. In some aspects, the control circuitry of the applicator (e.g., a microcontroller unit) can establish (step 2402) communication with a communication interface of cartridge 50, such as an NFC/RFID tag or other communication module, and read cartridge details including a unique cartridge identifier, authentication credential, status flags, and associated metadata stored in onboard memory. In some aspects, the system can determine whether a maximum number of read attempts has been exceeded (step 2404) and if the limit is reached, the MCU enters a low-power or sleep state until reactivated (step 2406). If the threshold is not reached, the process proceeds to determine (step 2418) whether a cartridge is installed and properly seated. If no cartridge is detected, the MCU returns to monitoring cartridge presence and position at until installation is confirmed.

Upon detecting a seated cartridge, the cartridge can be authenticated (step 2410) by retrieving and verifying an authenticity credential and associated digital signature from the cartridge's encrypted data set. If authentication fails, the system presents a “Load Authentic Cartridge” notification (step 2408) on an associated graphical user interface (e.g., on the applicator or a user device) and inhibits further configuration. If authentication succeeds, the process advances (step 2412) to evaluate reuse status by checking a non-reversible usage indicator, such as an irreversible usage flag written to a one-time-programmable or WORM memory element. If the usage flag indicates prior use or end-of-life, the system displays a “Load Unused Cartridge” notification (step 2414) and prevents activation.

For unused, authenticated cartridges, the process proceeds to validate compatibility (step 2422), including checking cartridge type, firmware or profile version, and feature support against the applicator's capabilities. If compatibility checks fail, a “Load Appropriate Cartridge” notification can be displayed (step 2420). In some aspects, operations can include determining whether the cartridge ID is indicative of the contents thereof being antiseptic (step 2424), including stem cells or some other biologic (step 2426), and/or being configured for electrospinning (step 2428).

If compatible, the MCU retrieves the cartridge's encrypted data set (step 2430), decrypts, and parses operating parameters including spray-generation parameters (e.g., flow rate, pulse width, pulse frequency, fluidic pressure set-point) and electrostatic-charging parameters (e.g., applied voltage magnitude, current limit, discharge waveform, polarity), together with formulation properties (e.g., viscosity range, conductivity) and optional nozzle-library references. In some aspects, the system can, based in part on the sensed decrypted information of the cartridge 50, select a nozzle geometry or nozzle profile if indicated by the decrypted data (step 2432), for example, choosing from a library optimized for different rheological profiles and viscosity ranges, and binds material specific and nozzle specific parameters into a working configuration. Subsystems can be configured according to the derived operating parameters (step 2434), including setting motor drive targets to achieve commanded flow rate, establishing air intake or pump rate, and setting the high voltage module's voltage level, polarity, and waveform. In some aspects, during this configuration, the graphical user interface can present information such as cartridge ID, content type, volume, applied voltage, flow rate, intended spray distance, read status, and write status, and may expose a start control once initialization succeeds. Other aspects of a respective cartridge can also be identified and evaluated in these example processes. For example, other solution types can be analyzed as well as cartridge origin and destination, whether its contents remain fully sealed, whether the cartridge has been loaded into the applicator previously, a prescribing entity (e.g., whether a doctor or other operator has requested the cartridge), whether the cartridge is associated with a particular patient or intended application.

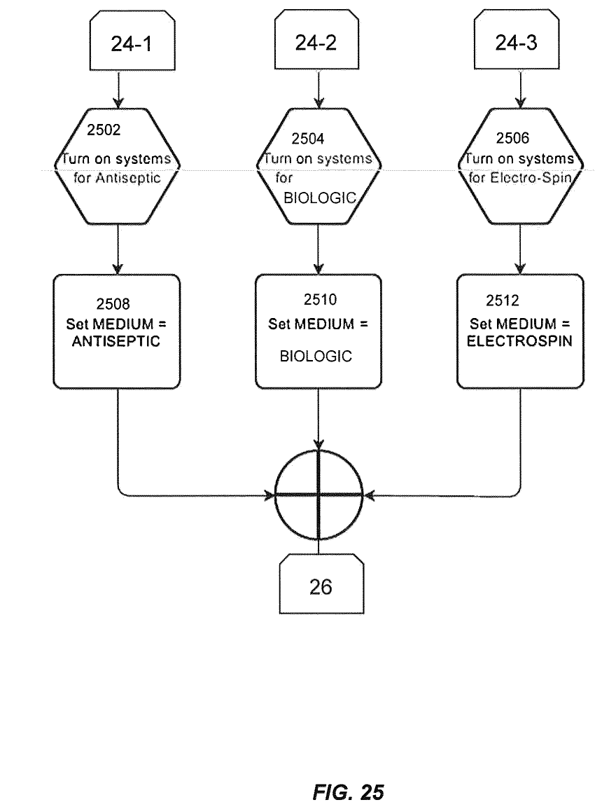

FIG. 25 illustrates a continuation of the cartridge authentication and initialization sequence described in FIG. 24. Following successful detection, authentication, and compatibility checks in FIG. 24, the encrypted data set can be retrieved and decrypted and a cartridge class or application mode determined, after which the process branches to cartridge-specific initialization paths 24-1, 24-2, or 24-3, corresponding by way of example to antiseptic delivery, biologic suspension (e.g., stem-cell or antibody formulations), and electrospinning operation, respectively. For example, the process of FIG. 25 can include step 2502, which can include the cartridge being identified as an antiseptic formulation and configuring subsystems using operational parameters obtained from the decrypted data set, including spray-generation settings such as commanded flow rate, pulse width, pulse frequency, and fluidic pressure set-point, and electrostatic-charging settings such as applied voltage magnitude, current limit, discharge waveform, polarity, etc.

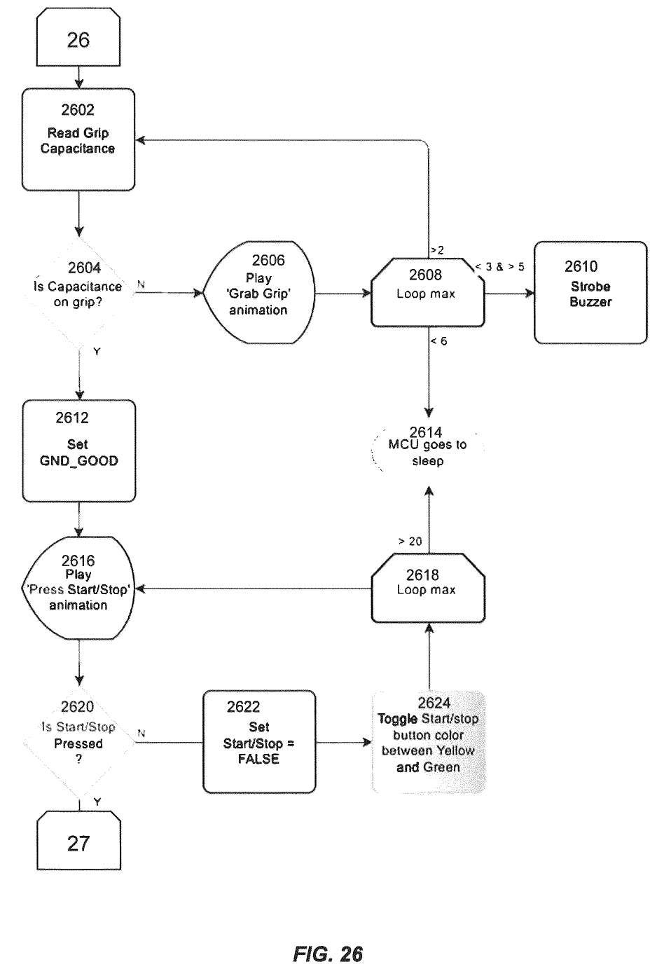

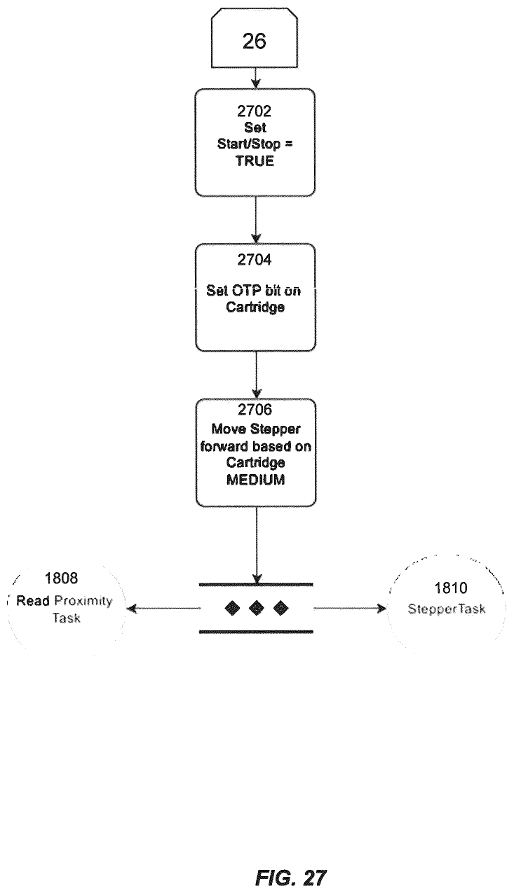

A nozzle geometry can be selected from a stored library referenced by the cartridge data and bind nozzle-specific and material-specific parameters to achieve a target plume geometry, droplet-size distribution, and charge-to-mass ratio appropriate for antiseptic application, etc. At step 2504, the process can include the cartridge being identified as a biologic suspension or related medium. In turn, formulation-appropriate controls derived from the decrypted parameters are identified for system configuration, including precise low-shear flow profiles, constrained voltage ranges, and any formulation-specific timing or pulsing profiles, while optionally enforcing temperature, sterility, or handling safeguards if indicated. Based in part on this data, a nozzle profile can be selected and/or verified as suitable for the viscosity and rheology encoded in the encrypted data (e.g., within a 1 cP to 10,000 cP range) At step 2506, the cartridge has been identified in an electrospinning mode so that the system configures high-voltage and feed parameters suitable for fiber formation, including voltage magnitude and waveform, flow rate and pulse strategy, and any auxiliary controls such as air assist, standoff distance, or collector bias as indicated by the decrypted data. In some aspects, nozzle-library selections and material-specific limits can be applied to promote stable jet formation and desired fiber characteristics. The process can also initiate system settings in steps 2508 (the operating medium parameter is set to ANTISEPTIC), 2510 (the operating medium parameter set to BIOLOGIC), 2512 (operating medium parameter set to ELECTROSPIN), respectively, and then proceed with remaining operational tasks previously described in process 1800 of FIG. 18, as further illustrated in FIGS. 26 and 27.

For example, in FIG. 27, start/stop can be set (step 2702), the OTP bit on the cartridge loaded with applicator 100 can be set (step 2704), and then the stepper of the applicator can be moved forward depending on the contents (i.e. the medium of the contents of the respective cartridge) (step 2706). Then, the proximity task 1808 can be read and if satisfactory, then the stepper task 1810 can be executed. In some aspects, the result of proximity task 1808 can be presented on a display screen of interface 87 and/or presented in one or more LEDs associated therewith.

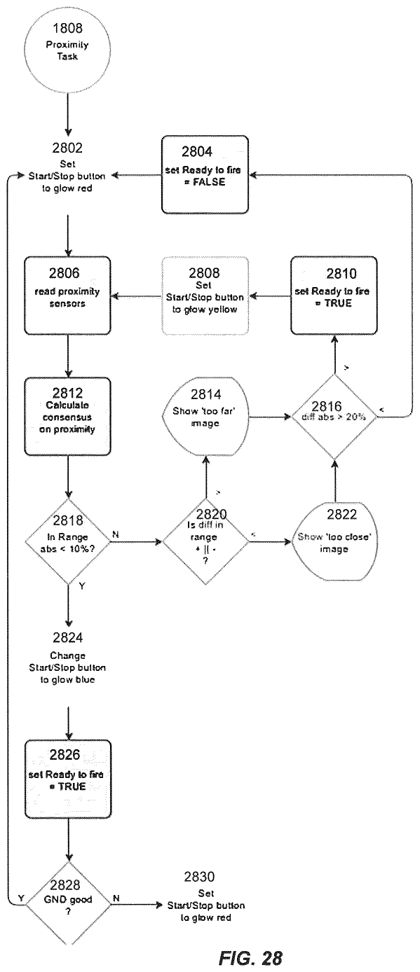

FIG. 28 is a flow chart of an exemplary process proximity task 1808 of previous process 1800 as to sensing and managing operations based on readings and related calculations from one or more proximity sensor of applicator 100.

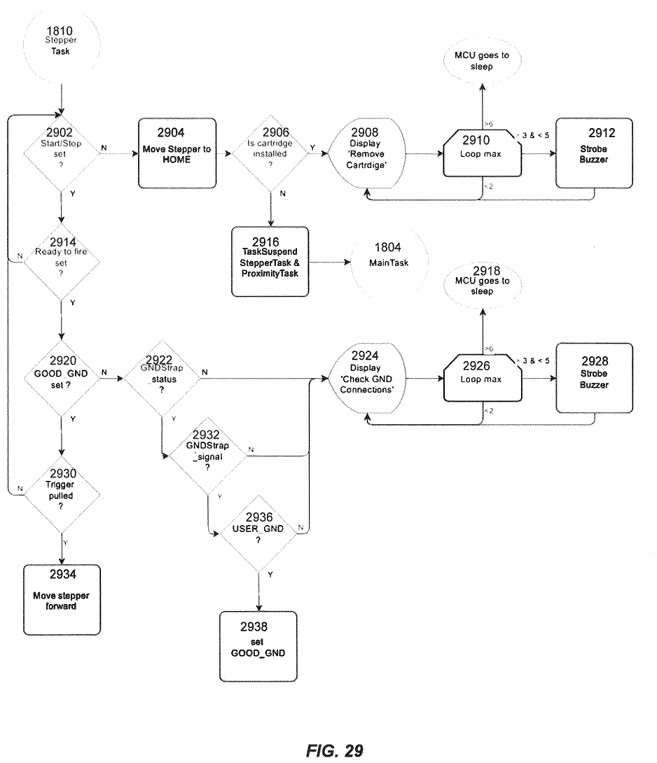

FIG. 29 is a flow chart of an exemplary process stepper task 1810 of previous process 1800 as to operations of the stepper motor of applicator 100 in connection with emitting contents from a cartridge loaded therewith towards a target site.

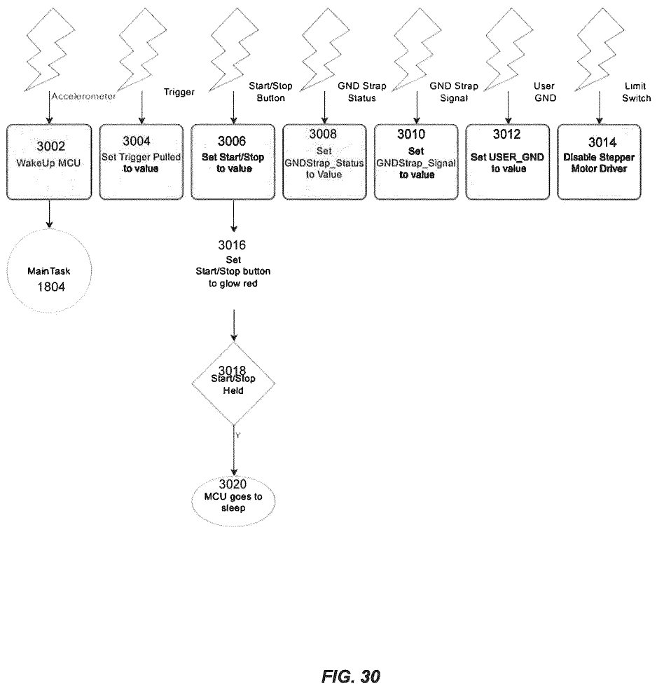

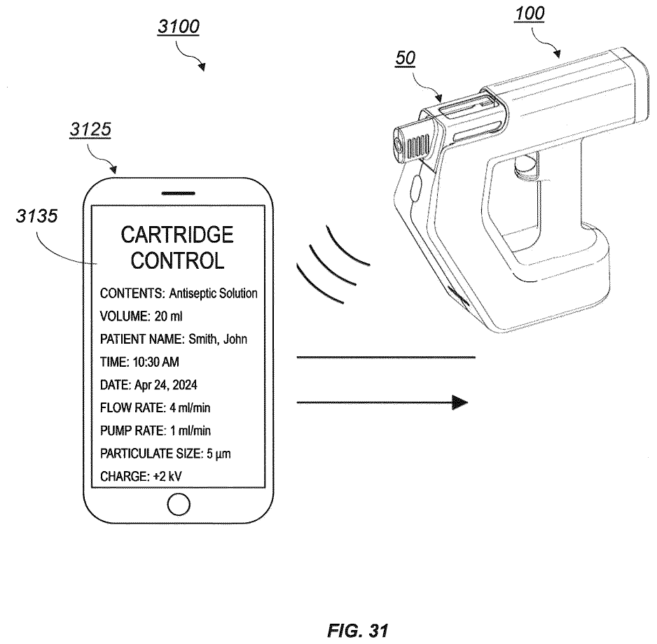

FIG. 30 is a flow chart of an exemplary processes which can be used to prior to executing main task 1804 and/or having the MCU enter a sleep mode (step 3020). For example, processes can include managing feedback from an accelerometer, manual trigger, actuation of a start/stop button, status of a ground strap, signal from a ground strap, presence of a user ground, as well as feedback from a limit switch.

Referring now to FIG. 31, a system 3100 is illustrated that includes applicator 100 and cartridge 50 assembled therewith. As described previously, when the cartridge 50 is removably inserted into the cartridge chamber, applicator 100 aligns electrical, pneumatic, and mechanical interfaces for dispensing and charging a treatment solution of cartridge 50. Cartridge 50 further includes a communication interface system having one or more communication or identification modules (e.g., near-field communication (NFC), radio-frequency identification (RFID), optical code readers such as QR, Bluetooth Low Energy (BLE), Wi-Fi, or other wireless or optical technologies) and an associated memory. The memory can store an encrypted data set that includes operating parameters, such as spray-generation parameters (e.g., flow rate, pulse width, pulse frequency, fluidic pressure set-point), electrostatic-charging parameters (e.g., applied voltage magnitude, current limit, discharge waveform, polarity), an authenticity credential, and the like. Executable instructions, when executed by one or more processors, cause the communication interface system to exchange data with an electrostatic spray-generation subsystem of applicator 100, retrieve and decrypt the encrypted data set, authenticate the cartridge 50, and/or configure applicator 100 in accordance with parameters obtained from the decrypted data.

In FIG. 31, the communication interface system of the cartridge 50 communicates with a user device 3125, which can be a mobile computing device (e.g., a smartphone) or any computing device (e.g., a personal computer). In some aspects, encrypted data and operational information can also be transmitted to and displayed on user interface 87 of applicator 100. User device 3125 presents an example user interface 3135, which along with user interface 87, can display information detected or received by the device 3125, including, for example, content type of the treatment solution, remaining volume, patient name, time and date, and operational parameters such as flow rate, pump rate, particulate or droplet size for a specific application (e.g., user condition, wound type, location of treatment site, and electrostatic charge parameters). User interface 3135 can also present cartridge authentication status, status of parameter execution, warning messages, and prompts for user intervention.

In certain implementations, sensors integrated with applicator 100 and/or cartridge 50 can measure operating and environmental conditions (e.g., ambient humidity, ambient temperature, applied voltage, motor speed, intake air flow, and air-flow velocity). Based on sensor feedback and/or retrieved information, applicator 100 can dynamically adjust one or more spray-generation and electrostatic-charging parameters (e.g., motor speed, air intake, applied voltage, etc.) to maintain a desired plume and/or droplet characteristics. In some examples, after a predetermined eject-mass or elapsed-time threshold is reached, the communication interface system 3150 writes a non-reversible usage flag (e.g., to a one-time-programmable or WORM memory) to inhibit subsequent reuse of the cartridge 50, and logs cartridge identification, usage metrics, and environmental data to tamper-resistant memory for post-procedural traceability. Detection of an irreversible usage flag can disable operation or activation of applicator 100.

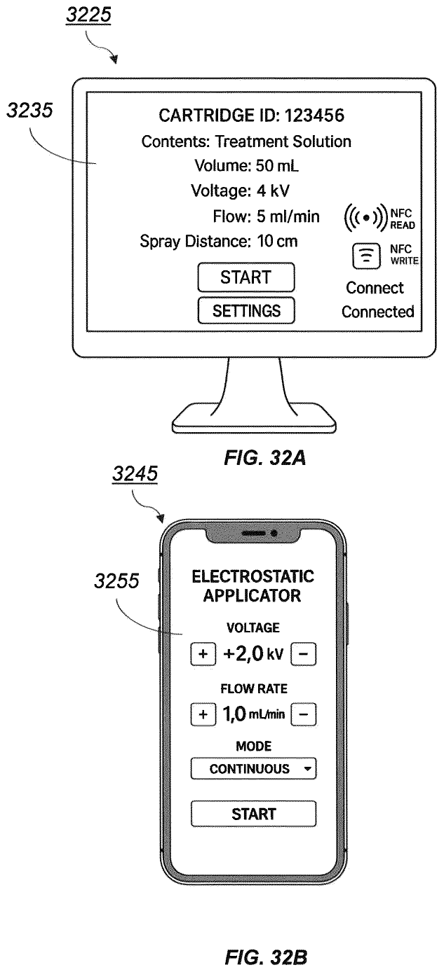

Referring now to FIG. 32A, an example user device 3225 is illustrated as a example computing device configured to communicate with an associated cartridge via a communication interface system and to present a user interface 3235 for setup, monitoring, control, and/or the like. In some examples, user interface 3235 can display cartridge identification information, content type of the treatment solution, remaining volume, applied voltage as a current value, target set-point, flow rate as a current value and/or target set-point, an intended spray distance indicative of a recommended standoff or application range, a start button to initiate a dispensing cycle, a settings button to access configuration panels, and/or the like. In some examples, interface 3235 can present a read status indicating a status (e.g., success or failure of data retrieval from cartridge 50, etc.) with a timestamp of a last read, and a write status indicating the success or failure of parameter updates or logging to cartridge 50 with a timestamp of the last write, present authentication results, warnings, prompts for user intervention, and the like. In turn, interface 3235 can allow a user to verify cartridge authenticity, confirm parameter execution, and proceed with treatment solution administration under validated conditions. In some examples, operations accessible via the interface 3235 include configuration and display of electrospray parameters and electrospinning parameters, selection or adjustment of spray pattern and plume geometry, and presentation of ancillary settings such as charge polarity, pulse width and frequency, and formulation-specific presets.

Referring now to FIG. 32B, an example user device 3245 is illustrated as a mobile computing device, such as a smartphone, that presents a user interface 3255 optimized for handheld operation and real-time management of operational parameters. In some examples, user interface 3255 includes applied voltage controls, flow rate controls, and an application mode selector that enables continuous, pulsed, electrospray and/or electrospinning operation, start and stop actuation controls to initiate and halt dispensing, together with indicators that confirm successful parameter application and reflect current read and write status. In some examples, interface 3255 can display cartridge identification, content type, remaining volume, intended spray distance, and other telemetry such as measured pump rate, intended droplet size for electrospray, or intended fiber diameter for electrospinning, and constrains user-adjustable set-points to limits encoded in the encrypted data set to ensure that adjustments remain within validated bounds for the specific formulation and nozzle geometry.

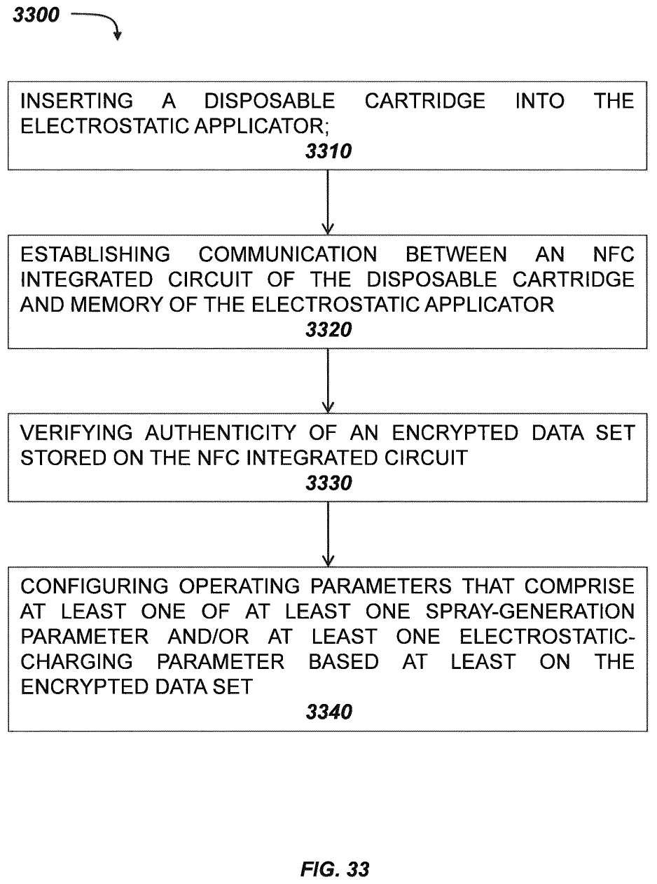

FIG. 33 is a flow diagram for a method 3300 of operating an example electrostatic applicator system. Step 3310 of method 3300 can include inserting a first disposable cartridge into an electrostatic applicator. Step 3320 of method 3300 can include establishing communication between an NFC integrated circuit of the disposable cartridge and memory of the electrostatic applicator. Step 3330 of method 3300 can include verifying authenticity of an encrypted data set stored on the NFC integrated circuit. Step 3340 of method 3300 can include configuring operating parameters that comprise at least one of at least one spray-generation parameter and/or at least one electrostatic-charging parameter based at least on the encrypted data set.

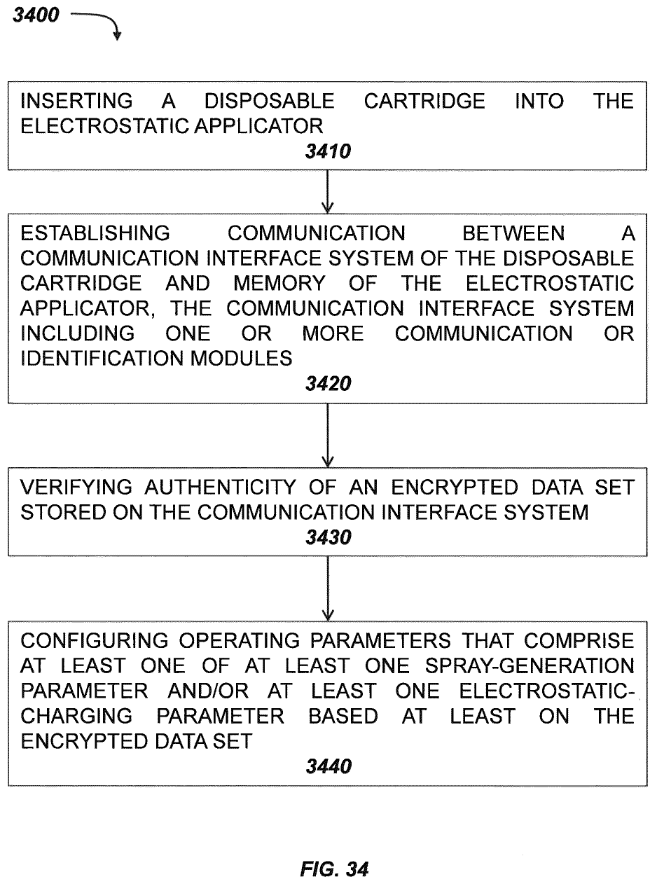

FIG. 34 is a flow diagram for a method 3400 of operating an example electrostatic applicator system. Step 3410 of method 3400 can include inserting a first disposable cartridge into an electrostatic applicator. Step 3320 of method 3300 can include establishing communication between an NFC integrated circuit of the disposable cartridge and memory of the electrostatic applicator. Step 3330 of method 3300 can include verifying authenticity of an encrypted data set stored on the NFC integrated circuit. Step 3340 of method 3300 can include configuring operating parameters that comprise at least one of at least one spray-generation parameter and/or at least one electrostatic-charging parameter based at least on the encrypted data set.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
1. A disposable fluid delivery system for an electrostatic applicator, including:
   a disposable cartridge containing and/or configured to be filled with or otherwise receive a treatment solution and a nozzle assembly configured to dispense the treatment solution;
   an communication interface system including memory and positioned with the cartridge, the communication interface system storing an encrypted data set including operating parameters that include at least one of (i) at least one spray-generation parameter, (ii) at least one electrostatic-charging parameter, and (iii) an authenticity credential, wherein the operating parameters are programmed and/or tuned based in part on properties of the treatment solution of the cartridge, wherein the memory includes instructions that, when executed by one or more processors, cause the communication interface system to:
      exchange data with an electrostatic spray-generation subsystem;
      retrieve and decrypt the encrypted data set;
      authenticate the disposable cartridge based on an authenticity credential of the disposable cartridge; and
      configure the electrostatic spray-generation subsystem in accordance with detected operating parameters of the encrypted data.
2. The system of Clause 1, wherein the disposable cartridge is configured to receive a syringe containing the treatment solution.
3. The system of Clause 1, wherein the disposable cartridge is removably attachable to a housing of the electrostatic applicator.
4. The system of Clause 1, wherein the disposable cartridge includes a fluid reservoir containing a treatment solution.
5. The system of Clause 1, wherein the disposable cartridge includes memory including information associated with operational parameters of the treatment solution.
6. The system of Clause 1, wherein the cartridge or a syringe assembled with the cartridge is configured to be filled with the treatment solution.
7. The system of Clause 1, wherein the instructions cause the communication interface system to write, after a predetermined eject-mass or elapsed-time threshold is reached, a non-reversible usage flag to the communication interface system to disable disabling subsequent reuse of the cartridge.
8. The system of Clause 1, wherein the communication interface system is an NFC integrated circuit is a one-time-programmable or write-once, read-many (WORM) device that prevents alteration of the encrypted data set.
9. The system of Clause 1, wherein the spray-generation parameter includes at least one of flow rate, pulse width, pulse frequency, and fluidic pressure set-point.
10. The system of Clause 1, wherein the electrostatic-charging parameter includes at least one of applied voltage magnitude, current limit, discharge waveform, and polarity.
11. The system of Clause 1, wherein the communication interface system is an NFC integrated circuit that is part of an NFC tag that includes an antenna configured to wirelessly communicate information.
12. The system of Clause 11, wherein the instructions cause the NFC integrated circuit to select from a library of nozzle geometries optimized for different rheological profiles of one or more treatment solutions.
13. The system of Clause 11, wherein the instructions cause the NFC integrated circuit to store nozzle-specific and material-specific parameters so that spray performance is tuned for a dispensing combination of nozzle and formulation parameters.
14. The system of Clause 1, further including a plurality of sensors configured to measure operating parameters of the cartridge including at least one of ambient humidity, motor speed, applied voltage, intake of air, ambient temperature, and air-flow velocity.
15. The system of Clause 14, wherein the instructions cause the communication interface system to dynamically adjust at least one of the spray-generation and electrostatic-charging parameter based on sensor feedback.
16. The system of Clause 14, wherein the instructions cause the communication interface system to dynamically adjust at least one the motor speed, the intake of air, and the applied voltage based on retrieved information.
17. The system of Clause 1, wherein the instructions cause the communication interface system to log cartridge identification, usage metrics, and environmental data to a tamper-resistant memory for post-procedural traceability.
18. An electrostatic applicator system for delivering a treatment solution to a target site, including:
   a portable reusable electrostatic applicator including:
   a device housing configured to be handheld;
   a motor in the device housing configured to drive a piston;
   a voltage source in the device housing;
   a high voltage module electrically connected to the voltage source; and
   a cartridge chamber; and
   a disposable cartridge removably insertable in the cartridge chamber, the disposable cartridge including:
      a nozzle assembly and a nozzle housing including an air supply port, a voltage port, and a delivery outlet;
      a cartridge housing at least partially enclosing the nozzle housing, a voltage wire, and a syringe; and
      a communication interface system integrated circuit including memory, the communication interface system storing an encrypted data set including operating parameters that include at least one of (i) at least one spray-generation parameter, (ii) at least one electrostatic-charging parameter, and (iii) an authenticity credential, wherein the operating parameters are programmed and/or tuned based in part on properties of the treatment solution of the cartridge; and
      wherein the memory includes instructions that, when executed by one or more processors, cause the communication interface system to:
         exchange data with an electrostatic spray-generation subsystem;
         retrieve and decrypt the encrypted data set;
         authenticate the disposable cartridge based on an authenticity credential of the disposable cartridge; and
         configure the electrostatic spray-generation subsystem in accordance with detected operating parameters of the encrypted data.
19. The system of Clause 18, further including: a graphical user interface that visually indicates at least one of cartridge authentication, status of parameter execution, and user intervention status.
20. The system of Clause 18, further including: a graphical user interface that visually displays a warning message.
21. The system of Clause 18, further including: a graphical user interface that visually presents information read from the communication interface system.
22. The system of Clause 18, wherein the disposable cartridge is configured to receive a syringe containing the treatment solution.
23. The system of Clause 18, wherein the disposable cartridge includes a fluid reservoir containing a treatment solution.
24. The system of Clause 18, wherein the disposable cartridge includes memory including information associated with operational parameters of the treatment solution.
25. The system of Clause 18, wherein the cartridge or a syringe assembled with the cartridge is configured to be filled with the treatment solution.
26. The system of Clause 18, wherein the instructions cause the communication interface system to write, after a predetermined eject-mass or elapsed-time threshold is reached, a non-reversible usage flag to the communication interface system to disable disabling subsequent reuse of the cartridge.
27. The system of Clause 18, wherein the communication interface system is an NFC integrated circuit is a one-time-programmable or write-once, read-many (WORM) device that prevents alteration of the encrypted data set.
28. The system of Clause 18, wherein the spray-generation parameter includes at least one of flow rate, pulse width, pulse frequency, and fluidic pressure set-point.
29. The system of Clause 18, wherein the electrostatic-charging parameter includes at least one of applied voltage magnitude, current limit, discharge waveform, and polarity.
30. The system of Clause 18, wherein the communication interface system includes an NFC integrated circuit is part of an NFC tag that includes an antenna configured to wirelessly communicate information.
31. The system of Clause 18, wherein the instructions cause the communication interface system to select from a library of nozzle geometries optimized for different rheological profiles of one or more treatment solutions.
32. The system of Clause 18, wherein the instructions cause the communication interface system to store nozzle-specific and material-specific parameters so that spray performance is tuned for a dispensing combination of nozzle and formulation parameters.
33. The system of Clause 18, further including a plurality of sensors configured to measure operating parameters of the cartridge including at least one of ambient humidity, motor speed, applied voltage, intake of air, ambient temperature, and air-flow velocity.
34. The system of Clause 18, wherein the instructions cause the communication interface system to log cartridge identification, usage metrics, and environmental data to a tamper-resistant memory for post-procedural traceability.
35. A method of operating disposable fluid delivery system for an electrostatic applicator, including:
   inserting a disposable cartridge into the electrostatic applicator;
   establishing communication between a communication interface system of the disposable cartridge and memory of the electrostatic applicator;
   verifying authenticity of an encrypted data set stored on the communication interface system; and
   configuring operating parameters that include at least one of at least one spray-generation parameter and/or at least one electrostatic-charging parameter based at least on the encrypted data set.
36. The method of Clause 35, further including: dispensing the treatment solution through a nozzle assembly of the disposable cartridge while applying electrostatic charge according to the configured operating parameters.
37. The method of Clause 35, further including: monitoring, using at least one or more sensors of the disposable cartridge and/or the electrostatic applicator and the communication interface system, cumulative dispensed mass or time of operation of treatment solution dispensed through the cartridge.
38. The method of Clause 35, further including: measuring, using at least one or more sensors, at least one of ambient humidity, motor speed, applied voltage, intake air flow, ambient temperature, or air-flow velocity of treatment solution dispensing through the disposable cartridge.
39. The method of Clause 35, further including: upon reaching a predefined end-of-life criterion, writing, using at least the communication interface system, an irreversible usage flag to inhibit further dispensing by the disposable cartridge.
40. The method of Clause 39, further including: disabling operation or activation of the electrostatic applicator upon detection of the irreversible usage flag.
41. The method of Clause 35, further including: adjusting the configured operating parameters during dispensing in response to environmental measurements of at least one or more sensors of the disposable cartridge and/or the electrostatic applicator.
42. The method of Clause 35, further including: recording an event log that includes cartridge identifier, environmental conditions, and parameter adjustments, the event log being cryptographically signed and stored in secure memory.
43. The method of Clause 35, further including:
   wirelessly communicating, using at least an NFC integrated circuit of the communication interface system of an NFC tag, cartridge operational information to a graphical user interface.
   44. The method of Clause 43, wherein the NFC integrated circuit is a one-time-programmable or write-once read-many (WORM) device that inhibits alteration of the encrypted data set after initial programming.
45. The method of Clause 35, wherein the at least one spray-generation parameter includes at least one of flow rate, pulse width, pulse frequency, and fluidic pressure set-point the spray-generation parameter includes at least one of flow rate, pulse width, pulse frequency, and fluidic pressure set-point.
46. The method of Clause 35, further including: selecting, in response to data received from the communication interface system, a nozzle geometry from a library of nozzle geometries for differing profiles of treatment solutions.
47. The method of Clause 35, further including: producing, using at least a nozzle assembly of the disposable cartridge, an electrostatically charged spray plume having a predetermined average droplet diameter and/or charge-to-mass ratio established by the configured spray-generation and electrostatic-charging parameters.
48. The method of Clause 35, wherein the configuring the operating parameters includes setting a voltage level that is modulated according to a flow rate commanded by the at least one spray-generation parameter.
49. A non-transitory computer-readable medium storing instructions that, when executed by processor, cause the processor to perform a method for operating an electrostatic applicator, the method including:
   establishing communication between an NFC integrated circuit of an disposable cartridge and memory of the electrostatic applicator;
   verifying authenticity of an encrypted data set stored on the NFC integrated circuit; and
   configuring operating parameters that include at least one of at least one spray-generation parameter and at least one electrostatic-charging parameter based at least on the encrypted data set.
50. The non-transitory computer-readable medium of Clause 49, wherein the communication is established between the NFC integrated circuit and memory by inserting the disposable cartridge into a chamber of the applicator.
51. The non-transitory computer-readable medium of Clause 49, wherein the encrypted data are derived from a statistical optimization modeling based in part on a droplet size distribution, a plume shape, and/or a mass-to-target metrics for a specific treatment solution.
52. The non-transitory computer-readable medium of Clause 49, wherein a treatment solution of the disposable cartridge includes a biologic suspension including at least one monoclonal antibodies, polyclonal antibodies, peptides, and combinations thereof.
53. The non-transitory computer-readable medium of Clause 49, wherein a nozzle assembly orientation of the disposable cartridge is selected to accommodate a viscosity range between 1 cP and 10,000 cP.
54. The non-transitory computer-readable medium of Clause 49, wherein the method performed includes dispensing a treatment solution through a nozzle assembly of the disposable cartridge while applying electrostatic charge according to the configured operating parameters.
55. The non-transitory computer-readable medium of Clause 49, wherein the method performed includes monitoring, using at least one or more sensors of the disposable cartridge and/or the electrostatic applicator and the NFC integrated circuit, cumulative dispensed mass or time of operation of treatment solution dispensed through the cartridge.
56. The non-transitory computer-readable medium of Clause 49, wherein the method performed includes measuring, using at least one or more sensors, at least one of ambient humidity, motor speed, applied voltage, intake air flow, ambient temperature, or air-flow velocity of treatment solution dispensing through the disposable cartridge.
57. The non-transitory computer-readable medium of Clause 49, wherein the method performed includes upon reaching a predefined end-of-life criterion, writing, using at least the NFC integrated circuit, an irreversible usage flag to inhibit further dispensing by the disposable cartridge.
58. The non-transitory computer-readable medium of Clause 49, wherein the method performed includes disabling operation or activation of the electrostatic applicator upon detection of an irreversible usage flag.
59. The non-transitory computer-readable medium of Clause 49, wherein the method performed includes adjusting the configured operating parameters during dispensing in response to environmental measurements of at least one or more sensors of the disposable cartridge and/or the electrostatic applicator.
60. The non-transitory computer-readable medium of Clause 49, wherein the method performed includes recording an event log that includes cartridge identifier, environmental conditions, and parameter adjustments, the event log being cryptographically signed and stored in secure memory.
61. The non-transitory computer-readable medium of Clause 49, wherein the method performed includes wirelessly communicating, using at least the NFC integrated circuit of an NFC tag, cartridge operational information to a graphical user interface.
62. The non-transitory computer-readable medium of Clause 49, wherein the method performed includes selecting, in response to data received from the NFC integrated circuit, a nozzle geometry from a library of nozzle geometries for differing profiles of treatment solutions.
63. The non-transitory computer-readable medium of Clause 49, wherein the method performed includes producing, using at least a nozzle assembly of the disposable cartridge, an electrostatically charged spray plume having a predetermined average droplet diameter and/or charge-to-mass ratio established by the configured spray-generation and electrostatic-charging parameters.
64. The non-transitory computer-readable medium of Clause 49, wherein the configuring the operating parameters includes setting a voltage level that is modulated according to a flow rate commanded by the at least one spray-generation parameter.
65. A disposable fluid delivery system for an electrostatic applicator, including:
a disposable cartridge containing and/or configured to be filled with or otherwise receive a treatment solution and a nozzle assembly configured to dispense the treatment solution;
a communication interface system coupled with the disposable cartridge, the communication interface system including one or more communication or identification modules, wherein the communication interface system includes a memory storing an encrypted data set including operating parameters that include at least one of at least one spray-generation parameter, at least one electrostatic-charging parameter, and an authenticity credential, wherein the memory includes executable instructions that, when executed by one or more processors, cause the communication interface system to:
   exchange data with an electrostatic spray-generation subsystem;
   retrieve and decrypt the encrypted data set;
   authenticate the disposable cartridge based on the authenticity credential; and
   configure the electrostatic spray-generation subsystem in accordance with the operating parameters obtained from the decrypted data set.
66. The system of Clause 65, wherein the communication module includes at least one of near-field communication (NFC), radio-frequency identification (RFID), optical code readers including QR code readers, Bluetooth® Low Energy (BLE), Wi-Fi, or other wireless or optical communication technologies.
67. The system of Clause 65, wherein the disposable cartridge is configured to receive a syringe containing the treatment solution.
68. The system of Clause 65, wherein the disposable cartridge is removably attachable to a housing of the electrostatic applicator.
69. The system of Clause 65, wherein the disposable cartridge includes a fluid reservoir containing a treatment solution.
70. The system of Clause 65, wherein the disposable cartridge includes memory including information associated with operational parameters of the treatment solution.
71. The system of Clause 65, wherein the cartridge or a syringe assembled with the cartridge is configured to be filled with the treatment solution.
72. The system of Clause 65, wherein the instructions cause the communication interface system to write, after a predetermined eject-mass or elapsed-time threshold is reached, a non-reversible usage flag to the communication interface system to disable disabling subsequent reuse of the cartridge.
73. The system of Clause 65, wherein the communication interface system is a one-time-programmable or write-once, read-many (WORM) device that prevents alteration of the encrypted data set.
74. The system of Clause 65, wherein the spray-generation parameter includes at least one of flow rate, pulse width, pulse frequency, and fluidic pressure set-point.
75. The system of Clause 65, wherein the electrostatic-charging parameter includes at least one of applied voltage magnitude, current limit, discharge waveform, and polarity.
76. The system of Clause 65, wherein the instructions cause the communication interface system to select from a library of nozzle geometries optimized for different rheological profiles of one or more treatment solutions.
77. The system of Clause 65, wherein the instructions cause the communication interface system to store nozzle-specific and material-specific parameters so that spray performance is tuned for a dispensing combination of nozzle and formulation parameters.
78. The system of Clause 65, further including a plurality of sensors configured to measure operating parameters of the cartridge including at least one of ambient humidity, motor speed, applied voltage, intake of air, ambient temperature, and air-flow velocity, and wherein the instructions cause the communication interface to dynamically adjust at least one of the spray-generation and electrostatic-charging parameter based on sensor feedback.
79. The system of Clause 65, further including a plurality of sensors configured to measure operating parameters of the cartridge including at least one of ambient humidity, motor speed, applied voltage, intake of air, ambient temperature, and air-flow velocity, and wherein the instructions cause the communication interface to dynamically adjust at least one the motor speed, the intake of air, and the applied voltage based on retrieved information.
80. The system of Clause 65, wherein the instructions cause the communication interface system to log cartridge identification, usage metrics, and environmental data to a tamper-resistant memory for post-procedural traceability.

What is claimed is:

1. A method of operating disposable fluid delivery system for an electrostatic applicator, comprising:
establishing communication between a communication interface system of a disposable cartridge and memory of the electrostatic applicator;
verifying authenticity of an encrypted data set stored on the communication interface system; and
configuring operating parameters that comprise at least one of at least one spray-generation parameter and/or at least one electrostatic-charging parameter based at least on the encrypted data set.

2. The method of claim 1, further comprising: dispensing a treatment solution through a nozzle assembly of the disposable cartridge while applying electrostatic charge according to the configured operating parameters.

3. The method of claim 1, further comprising: monitoring, using at least one or more sensors of the disposable cartridge and/or the electrostatic applicator and the communication interface system, cumulative dispensed mass or time of operation of treatment solution dispensed through the cartridge.

4. The method of claim 1, further comprising: measuring, using at least one or more sensors, at least one of ambient humidity, motor speed, applied voltage, intake air flow, ambient temperature, or air-flow velocity of treatment solution dispensing through the disposable cartridge.

5. The method of claim 1, further comprising: upon reaching a predefined end-of-life criterion, writing, using at least the communication interface system, an irreversible usage flag to inhibit further dispensing by the disposable cartridge.

6. The method of claim 5, further comprising: disabling operation or activation of the electrostatic applicator upon detection of the irreversible usage flag.

7. The method of claim 1, further comprising: adjusting the configured operating parameters during dispensing in response to environmental measurements of at least one or more sensors of the disposable cartridge and/or the electrostatic applicator.

8. The method of claim 1, further comprising: recording an event log that comprises cartridge identifier, environmental conditions, and parameter adjustments, the event log being cryptographically signed and stored in secure memory.

9. The method of claim 1, further comprising:
wirelessly communicating, using at least a NFC integrated circuit of an NFC tag of the communication interface system, cartridge operational information to a graphical user interface.

10. The method of claim 1, wherein the at least one spray-generation parameter comprises at least one of flow rate, pulse width, pulse frequency, and fluidic pressure set-point the spray-generation parameter comprises at least one of flow rate, pulse width, pulse frequency, and fluidic pressure set-point.

11. The method of claim 1, further comprising: selecting, in response to data received from the communication interface system, a nozzle geometry from a library of nozzle geometries for differing profiles of treatment solutions.

12. The method of claim 1, further comprising: producing, using at least a nozzle assembly of the disposable cartridge, an electrostatically charged spray plume having a predetermined average droplet diameter and/or charge-to-mass ratio established by the configured spray-generation and electrostatic-charging parameters.

13. The method of claim 1, wherein the configuring the operating parameters comprises setting a voltage level that is modulated according to a flow rate commanded by the at least one spray-generation parameter.

14. A disposable fluid delivery system for an electrostatic applicator, comprising:
a disposable cartridge containing and/or configured to be filled with or otherwise receive a treatment solution and a nozzle assembly configured to dispense the treatment solution;
a communication interface system coupled with or part of the disposable cartridge, the communication interface system comprising one or more communication or identification modules, wherein the communication interface system comprises a memory storing an encrypted data set comprising operating parameters that comprise at least one of at least one spray-generation parameter, at least one electrostatic-charging parameter, and an authenticity credential, wherein the memory comprises executable instructions that, when executed by one or more processors, cause the communication interface system to:
exchange data with an electrostatic spray-generation subsystem;
retrieve and decrypt the encrypted data set;
authenticate the disposable cartridge based on the authenticity credential; and
configure the electrostatic spray-generation subsystem in accordance with the operating parameters obtained from the decrypted data set.

15. The system of claim 14, wherein the communication module comprises at least one of near-field communication (NFC), radio-frequency identification (RFID), optical code readers comprising QR code readers, Bluetooth® Low Energy (BLE), Wi-Fi, or other wireless or optical communication technologies.

16. The system of claim 14, wherein the disposable cartridge comprises memory comprising information associated with operational parameters of the treatment solution.

17. The system of claim 14, wherein the communication interface system is a one-time-programmable or write-once, read-many (WORM) device that prevents alteration of the encrypted data set.

18. The system of claim 14, wherein the spray-generation parameter comprises at least one of flow rate, pulse width, pulse frequency, and fluidic pressure set-point; and/or
wherein the electrostatic-charging parameter comprises at least one of applied voltage magnitude, current limit, discharge waveform, and polarity.

19. The system of claim 14, wherein the instructions cause the communication interface system to select from a library of nozzle geometries optimized for different rheological profiles of one or more treatment solutions.

20. A non-transitory computer-readable medium storing instructions that, when executed by processor, cause the processor to perform a method for operating an electrostatic applicator, the method comprising:
establishing communication between a communication interface system of a disposable cartridge and memory of the electrostatic applicator;
verifying authenticity of an encrypted data set stored on the communication interface system; and
configuring operating parameters that comprise at least one of at least one spray-generation parameter and/or at least one electrostatic-charging parameter based at least on the encrypted data set.
